(19) United States
(12) Patent Application Publication
(10) Pub. No.: US 2023/0398102 A1
(43) Pub. Date: Dec. 14, 2023

(54) RIFAXIMIN LIQUID FORMULATIONS FOR USE INTHE TREATMENT OF SICKLE CELL DISEASE

(71) Applicant: Bausch Health Ireland Limited, Dublin (IE)

(72) Inventors: Arturo J. Angel, Santa Rosa, CA (US); Kasturi R. Pawar, Rohnert Park, CA (US); Radhakrishnan S. Pillai, Santa Rosa, CA (US)

(21) Appl. No.: 18/034,411

(22) PCT Filed: Oct. 29, 2021
(86) PCT No.: PCT/EP2021/080168

§ 371 Date: Apr. 28, 2023

Related U.S. Application Data
(60) Provisional application No. 63/107,406, filed on Oct. 29, 2020.

(51) Int. Cl. A61K 31/437; A61K 47/44; A61K 47/14; A61K 47/26; A61K 47/08; A61K 31/198; A61K 31/17; A61K 31/485; A61P 7/06

(52) U.S. Cl. CPC A61K 31/437; A61K 47/44; A61K 47/14; A61K 47/26; A61K 47/08; A61K 31/198; A61K 31/17; A61K 31/485; A61P 7/06

(57) ABSTRACT

Pharmaceutical composition comprising rifaximin, a hydrogenated castor oil, and at least one additional solubilizing excipient for use in the treatment of sickle cell disease, vaso-occlusive crises or circulating ages neutrophils.

### RELATED APPLICATIONS

This application claims priority to U.S. Provisional Application No. 63/107,406, filed Oct. 29, 2020, the entire contents of which are incorporated herein by reference.

### BACKGROUND

Sickle cell disease (SCD) affects approximately 100,000 Americans and while the standard of care has improved, there remains an urgent need for new and efficacious SCD therapies.

### SUMMARY

Provided herein are treatments of sickle cell disease (SCD) that include the use of compositions as described herein, such as compositions comprising rifaximin, a hydrogenated castor oil, and at least one additional solubilizing excipient.

Patients having SCD may have recurrent painful vaso-occlusive crises (VOCs), which is the most common clinical manifestation of SCD. VOC occurs when the patient's microcirculation is obstructed by sickled red blood cells (RBCs), which may result in ischemic injury, ulcers, priapism, organ damage, and spontaneous abortion. Furthermore, patients having SCD may, overall, have a poor quality of life and a shortened lifespan.

Neutrophils have been implicated in regulating VOC in SCD patients. SCD patients with WBC>15×10⁹/L are more likely to develop stroke, acute chest syndrome, and premature death. Neutrophils in SCD patients are also shown to exhibit increased levels of activation molecules, including CD64 and CD11b/CD18, with their sera having elevated levels of soluble CD62L. A subset of neutrophils known as circulating aged neutrophils (CANs) are substantially elevated. CANs are characterized by having a high surface expression of CXCR4 and low CD62L. Activated and aged neutrophils may be immobilized in the circulatory system on the endothelium and form the nidus for the adhesion of sickled RBCs, which may lead to VOC.

It has been reported that modulating intestinal microbial composition may be a therapeutic option in treating SCD patients to reduce VOC through the reduction of activated and aged neutrophils. In one study, it was found that a 550 mg dose of rifaximin (i.e., XIFAXAN® 550 mg), delivered BID, was capable of reducing CANs in SCD patients (Clinical Trial Identifier: NCT03719729). Furthermore, when SCD patients received a 550 mg dose of rifaximin (i.e., XIFAXAN® 550 mg) BID for 6 months, the result was a decrease in the number of VOCs, and thus an increased quality of life.

The rifaximin compositions described herein are provided to increase the gastrointestinal luminal solubility of rifaximin, while minimizing systemic exposure. Accordingly, the rifaximin compositions described herein provide a therapy for treating SCD in a patient by, for example, and without being limited to any one theory of the invention, (1) reducing levels of elevated circulating aged neutrophils (CANs), and/or (2) reducing or preventing the occurrence of vaso-occlusive crises (VOCs). As opposed to prior therapies, and the knowledge in the art, the benefits offered by the invention described herein are provided at a substantially reduced dosage of rifaximin while providing clinical benefit.

In an embodiment, the invention described herein includes a method of treating sickle cell disease (SCD) in a patient in need thereof comprising administering a disclosed rifaximin composition to the patient. In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient. In some embodiments, treating vaso-occlusive crisis (VOC) in the patient comprises (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. In some embodiments, the method of treating sickle cell (SCD) in the patient comprises alleviating one or more symptoms of vaso-occlusive crisis (VOC) in the patient. In some embodiments, the method of treating sickle cell disease (SCD) in the patient comprises reducing or preventing the occurrence of vaso-occlusive crises (VOCs) in the patient. In some embodiments, the method of treating sickle cell disease (SCD) comprises reducing the duration or severity of VOC in the patient. In some embodiments, the method of treating sickle cell disease (SCD) in the patient comprises mediating or otherwise reducing the patient's opioid usage during vaso-occlusive crisis (VOC) in the patient.

In some embodiments, the methods described herein further include administering an additional therapeutic agent, such as an SCD therapeutic agent. In some embodiments, the additional therapeutic agent is an SCD therapeutic agent. In some embodiments, the SCD therapeutic agent is selected from the group consisting of hydroxyurea, L-glutamine, hydroxycarbamide, an erythropoietin stimulating agent, an opioid analgesic, and combinations thereof. In some embodiments, the opioid analgesic is selected from the group consisting of morphine, codeine, hydrocodone, hydromorphone, methadone, tramadol, oxycodone, tapentadol, fentanyl, and combinations thereof. In some embodiments, the SCD therapeutic agent comprises an opioid analgesic.

In an embodiment, the compositions described in the present methods comprise rifaximin, a hydrogenated castor oil, and at least one additional solubilizing excipient. In some embodiments, the compositions described herein include a low dose rifaximin. In some embodiments, the compositions described herein may be pharmaceutically acceptable compositions. In an embodiment, the invention described herein includes a pharmaceutically acceptable composition comprising low dose rifaximin, a hydrogenated castor oil, and at least one additional solubilizing excipient.

In some embodiments, the hydrogenated castor oil may be polyoxyl 40 hydrogenated castor oil (e.g., Cremophor® RH-40).

In some embodiments, the hydrogenated castor oil may be present in an amount ranging from about 25% to about 65% by weight of the composition.

In some embodiments, the hydrogenated castor oil may be present in an amount ranging from about 25% to about 50% by weight of the composition.

In some embodiments, the hydrogenated castor oil may be present in an amount ranging from about 30% to about 45% by weight of the composition.

In some embodiments, the hydrogenated castor oil may be present in an amount ranging from about 35% to about 40% by weight of the composition. In some embodiments, the hydrogenated castor oil may be present in an amount of about 35% or about 40% by weight of the composition.

In some embodiments, the hydrogenated castor oil may be present in an amount ranging from about 45% to about 65% by weight of the composition.

In some embodiments, the at least one additional solubilizing excipient may be a plurality of additional solubilizing excipients. In some embodiments, the at least one additional solubilizing excipient may be selected from the group consisting of a water-soluble organic solvent, a non-ionic surfactant, a water-insoluble lipid, long-chain triglycerides, and combinations thereof. In some embodiments, the at least one additional solubilizing excipient may comprise one or more of a water-soluble organic solvent, a non-ionic surfactant, a water-insoluble lipid, and long-chain triglycerides. In some embodiments, the at least one additional solubilizing excipient may be selected from the group consisting of polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, and combinations thereof. In some embodiments, the at least one additional solubilizing excipient may comprise one or more of polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, and olelyl alcohol.

In some embodiments, the at least one additional solubilizing excipient may be one or more additional solubilizing excipients that allows for or otherwise provides for a rifaximin saturation solubility of greater than about 10% by weight of the rifaximin in the composition. In some embodiments, the at least one additional solubilizing excipient may be one or more additional solubilizing excipients that allows for or otherwise provides for a rifaximin saturation solubility of greater than about 14% by weight of the rifaximin in the composition.

In some embodiments, the at least one additional solubilizing excipient may be selected from the group consisting of polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, polysorbate 20, oleic acid, caprylic capric triglycerides, propylene glycol, sesame oil, soybean oil, corn oil, and combinations thereof. In some embodiments, the at least one additional solubilizing excipient may comprise at least one of polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, polysorbate 20, oleic acid, caprylic capric triglycerides, propylene glycol, sesame oil, soybean oil, and corn oil.

In some embodiments, the at least one additional solubilizing excipient may be selected from the group consisting of castor oil, glyceryl caprylate, polysorbate 80, diethyl sebacate, diethylene glycol monoethyl ether, and combinations thereof. In some embodiments, the at least one additional solubilizing excipient may comprise one or more of castor oil, glyceryl caprylate, polysorbate 80, and diethyl sebacate.

In some embodiments, the at least one additional solubilizing excipient may be present in an amount ranging from about 25% to about 65% by weight of the composition.

In some embodiments, the at least one additional solubilizing excipient may be present in an amount ranging from about 30% to about 65% by weight of the composition.

In some embodiments, the at least one additional solubilizing excipient may be present in an amount ranging from about 35% to about 65% by weight of the composition.

In some embodiments, the at least on additional solubilizing excipient may be present in an amount ranging from about 40% to about 65% by weight of the composition.

In some embodiments, the at least one additional solubilizing excipient may be present in an amount ranging from about 45% to about 65% by weight of the composition.

In some embodiments, the at least one additional solubilizing excipient may be present in an amount ranging from about 50% to about 65% by weight of the composition.

In some embodiments, the at least one additional solubilizing excipient may be present in an amount ranging from about 50% to about 60% by weight of the composition.

In some embodiments, the at least one additional solubilizing excipient may be present in an amount ranging from about 55% to about 60% by weight of the composition.

In some embodiments, the at least one additional solubilizing excipient may be present in an amount ranging from about 55% to about 65% by weight of the composition.

In some embodiments, the at least one additional solubilizing excipient may be present in an amount ranging from about 60% to about 65% by weight of the composition.

In some embodiments, the at least one additional solubilizing excipient may be present in an amount ranging from about 62% to about 63% by weight of the composition.

In some embodiments, the at least one additional solubilizing excipient may be a combination of at least two additional solubilizing excipients. In some embodiments, the at least one additional solubilizing excipient may be a combination of at least three additional solubilizing excipients. In some embodiments, the at least one additional solubilizing excipient comprises castor oil and glyceryl caprylate. In some embodiments, the at least one additional solubilizing excipient comprises castor oil and polysorbate 80. In some embodiments, the at least one additional solubilizing excipient comprises glyceryl caprylate and polysorbate 80. In some embodiments, the at least one additional solubilizing excipient comprises castor oil, glyceryl caprylate, and polysorbate 80. In some embodiments, the at least one additional solubilizing excipient is a combination of castor oil, glyceryl caprylate, and polysorbate 80.

In some embodiments, the compositions described herein include castor oil in an amount ranging from about 5% to about 15% by weight of the composition; glyceryl caprylate in an amount ranging from about 5% to about 15% by weight of the composition; and polysorbate 80 in an amount ranging from about 20% to about 40% by weight of the composition.

In some embodiments, the compositions described herein include castor oil in an amount ranging from about 5% to about 15% by weight of the composition; glyceryl caprylate in an amount ranging from about 5% to about 15% by weight of the composition; and polysorbate 80 in an amount ranging from about 20% to about 40% by weight of the composition.

In some embodiments, the compositions described herein include castor oil in an amount ranging from about 8% to about 12% by weight of the composition; glyceryl caprylate in an amount ranging from about 10% to about 14% by weight of the composition; and polysorbate 80 in an amount ranging from about 30% to about 35% by weight of the composition.

In some embodiments, the compositions described herein include castor oil in an amount ranging from about 10% to about 20% by weight of the composition; glyceryl caprylate in an amount ranging from about 5% to about 15% by weight of the composition; and polysorbate 80 in an amount ranging from about 20% to about 40% by weight of the composition.

In some embodiments, the compositions described herein include castor oil in an amount ranging from about 13% to about 18% by weight of the composition; glyceryl caprylate in an amount ranging from about 10% to about 14% by weight of the composition; and polysorbate 80 in an amount ranging from about 30% to about 35% by weight of the composition.

In some embodiments, the compositions described herein include castor oil in an amount ranging from about 5% to about 15% by weight of the composition; glyceryl caprylate in an amount ranging from about 10% to about 15% by weight of the composition; and polysorbate 80 in an amount ranging from about 35% to about 45% by weight of the composition.

In some embodiments, the compositions described herein include castor oil in an amount ranging from about 8% to about 12% by weight of the composition; glyceryl caprylate in an amount ranging from about 10% to about 15% by weight of the composition; and polysorbate 80 in an amount ranging from about 38% to about 42% by weight of the composition.

In some embodiments, the at least one additional solubilizing excipient comprises diethyl sebacate and diethylene glycol monoethyl ether. In some embodiments, the at least one additional solubilizing excipient comprises diethyl sebacate and diethylene glycol monoethyl ether. In some embodiments, the at least one additional solubilizing excipient is a combination of diethyl sebacate and diethylene glycol monoethyl ether. In some embodiments, the at least one additional solubilizing excipient is a combination of diethyl sebacate and diethylene glycol monoethyl ether.

In some embodiments, the compositions described herein include diethyl sebacate in an amount ranging from about 20% to about 35% by weight of the composition; and diethylene glycol monoethyl ether in an amount ranging from about 20% to about 35% by weight of the composition. In some embodiments, the compositions described herein include diethyl sebacate in an amount ranging from about 20% to about 35% by weight of the composition; and diethylene glycol monoethyl ether in an amount ranging from about 20% to about 35% by weight of the composition.

In some embodiments, the compositions described herein include diethyl sebacate in an amount ranging from about 25% to about 30% by weight of the composition; and diethylene glycol monoethyl ether in an amount ranging from about 25% to about 30% by weight of the composition. In some embodiments, the compositions described herein include diethyl sebacate in an amount ranging from about 25% to about 30% by weight of the composition; and diethylene glycol monoethyl ether in an amount ranging from about 25% to about 30% by weight of the composition.

In some embodiments, the compositions described herein include diethyl sebacate in an amount ranging from about 10% to about 20% by weight of the composition; and diethylene glycol monoethyl ether in an amount ranging from about 30% to about 45% by weight of the composition. In some embodiments, the compositions described herein include diethyl sebacate in an amount ranging from about 10% to about 20% by weight of the composition; and diethylene glycol monoethyl ether in an amount ranging from about 30% to about 45% by weight of the composition.

In some embodiments, the compositions described herein include diethyl sebacate in an amount ranging from about 14% to about 18% by weight of the composition; and diethylene glycol monoethyl ether in an amount ranging from about 36% to about 40% by weight of the composition. In some embodiments, the compositions described herein include diethyl sebacate in an amount ranging from about 14% to about 18% by weight of the composition; and diethylene glycol monoethyl ether in an amount ranging from about 36% to about 40% by weight of the composition.

In some embodiments, the compositions described herein may include an antioxidant and/or chelating agent. In some embodiments, the compositions described herein may include an antioxidant and/or chelating agent selected from the group consisting of ascorbyl palmitate, butylated hydroxyanisole, butylated hydroxytoluene (BHT), citric acid, potassium metabisulfite, sodium metabisulfite, cysteine, propyl gallate, sodium thiosulfate, vitamin E, 3,4-dihydroxybenzoic acid, and a combination thereof. In some embodiments, the compositions described herein may include one or more of ascorbyl palmitate, butylated hydroxyanisole, butylated hydroxytoluene (BHT), citric acid, potassium metabisulfite, sodium metabisulfite, propyl gallate, sodium thiosulfate, cysteine, vitamin E, and 3,4-dihydroxybenzoic acid.

In some embodiments, the compositions described herein may include an antioxidant in an amount ranging from about 0.01% to about 0.1% by weight of the composition.

In some embodiments, the compositions described herein may include BHT.

In some embodiments, the compositions described herein may include BHT in an amount ranging from about 0.01% to about 0.1% by weight of the composition.

In some embodiments, the compositions described herein may include citric acid.

In some embodiments, the compositions described herein may include citric acid in an amount ranging from about 0.01% to about 0.1% by weight of the composition.

In some embodiments, the compositions described herein may include ascorbyl palmitate.

In some embodiments, the compositions described herein may include ascorbyl palmitate in an amount ranging from about 0.05% to about 0.15% by weight of the composition.

In some embodiments, the compositions described herein may include BHT and citric acid.

In some embodiments, the compositions described herein may include BHT and citric acid each in an amount ranging from about 0.01% to about 0.1% by weight of the composition.

In some embodiments, the compositions described herein may include BHT, citric acid, and ascorbyl palmitate.

In some embodiments, the compositions described herein may include BHT, citric acid, and ascorbyl palmitate, each in an amount ranging from about 0.05% to about 0.15% by weight of the composition.

In some embodiments, the compositions described herein may include rifaximin in an amount ranging from about 2.5% to about 15% by weight of the composition.

In some embodiments, the compositions described herein may include rifaximin in an amount ranging from about 2.5% to about 12% by weight of the composition.

In some embodiments, the compositions described herein may include rifaximin in an amount ranging from about 5% to about 10% by weight of the composition.

In some embodiments, the compositions described herein may include rifaximin in an amount ranging from about 1.0% to about 15% by weight of the composition.

In some embodiments, the compositions described herein may include rifaximin in an amount ranging from about 1.0% to about 5% by weight of the composition.

In some embodiments, the compositions described herein may include rifaximin in an amount that is less than about 125 mg. In some embodiments, the compositions described herein may include rifaximin in an amount ranging from about 10 mg to about 125 mg. In some embodiments, the compositions described herein may include rifaximin in an amount ranging from about 25 mg to about 125 mg. In some embodiments, the compositions described herein may include rifaximin in an amount ranging from about 25 mg to about 75 mg. In some embodiments, the compositions described herein may include rifaximin in an amount ranging from about 75 mg to about 125 mg. In some embodiments, the compositions described herein may include rifaximin in an amount of about 50 mg. In some embodiments, the compositions described herein may include rifaximin in an amount of about 100 mg.

In some embodiments, the compositions described herein may include rifaximin in an amount that is less than about 125 mg. In some embodiments, the compositions described herein may include rifaximin in an amount ranging from about 1 mg to about 50 mg. In some embodiments, the compositions described herein may include rifaximin in an amount ranging from about 1 mg to about 25 mg. In some embodiments, the compositions described herein may include rifaximin in an amount ranging from about 1 mg to about 10 mg. In some embodiments, the compositions described herein may include rifaximin in an amount ranging from about 1 mg to about 5 mg. In some embodiments, the compositions described herein may include rifaximin in an amount of about 2 mg to about 5 mg. In some embodiments, the compositions described herein may include rifaximin in an amount of about 3 mg to about 4 mg.

In an embodiment, the invention may include a composition comprising about 10% rifaximin, about 10% castor oil, about 12% glyceryl caprylate, about 33% polysorbate and about 35% polyoxyl 40 hydrogenated castor oil, by weight of the composition. In an embodiment, the invention may include a composition comprising about 10% rifaximin, about 10% castor oil, about 12% glyceryl caprylate, about 33% polysorbate 80, and about 35% polyoxyl 40 hydrogenated castor oil, by weight of the composition.

In an embodiment, the invention may include a composition comprising about 10% rifaximin, about 35% polyoxyl 40 hydrogenated castor oil, about 27.5% diethyl sebacate, and about 27.5% diethylene glycol monoethyl ether, by weight of the composition.

In an embodiment, the invention may include a composition comprising about 5% rifaximin, about 15% castor oil, about 12% glyceryl caprylate, about 33% polysorbate 80, and about 35% polyoxyl 40 hydrogenated castor oil, by weight of the composition.

In an embodiment, the invention may include a composition comprising about 10% rifaximin, about 40% polyoxyl 40 hydrogenated castor oil, about 16.5% diethyl sebacate, and about 38.5% diethylene glycol monoethyl ether, by weight of the composition.

In an embodiment, the invention may include a composition comprising about 2.5% rifaximin, about 10% castor oil, about 12% glyceryl caprylate, about 40% polysorbate and about 35% polyoxyl 40 hydrogenated castor oil, by weight of the composition.

In some embodiments, the compositions described herein are liquid compositions. In some embodiments, the compositions described herein may be formulated in a soft capsule dosage form. In some embodiments, the compositions described herein may be liquid compositions formulated in a soft capsule dosage form. In some embodiments, the compositions described herein may be formulated in a hard capsule dosage form. In some embodiments, the compositions described herein may be liquid compositions formulated in a hard capsule dosage form. In some embodiments, the compositions described herein may be formulated in a gelatin capsule dosage form. In some embodiments, the compositions described herein may be liquid compositions formulated in a gelatin capsule dosage form.

### BRIEF DESCRIPTION OF THE FIGURES

FIG. 1 shows the dissolution results for inventive composition 5425-67A compared with placebo (i.e., no rifaximin) 5425-67B, Xifaxan 550 mg (Opadry II), and 40 mg and 80 mg solid dispersion formulations as described in WO 2018/064472 (see also US Application Publication No. 2019-0224175, the entirety of which is incorporated herein by reference).

FIG. 2. shows the dissolution results for inventive composition 5425-66A compared with placebo (i.e., no rifaximin) 5425-66B, Xifaxan 550 mg (Opadry II), and 40 mg and 80 mg solid dispersion formulations as described in WO 2018/064472.

FIG. 3. shows the dissolution results for inventive composition 5425-68A at pH 7.4 compared with placebo (i.e., no rifaximin) 5425-68B, Xifaxan 550 mg (Opadry II), and mg and 80 mg solid dispersion formulations as described in WO 2018/064472.

FIG. 4. shows the dissolution results for inventive composition 5425-68A at pH 4.5 compared with placebo (i.e., no rifaximin) 5425-68B, Xifaxan 550 mg (Opadry II), and mg and 80 mg solid dispersion formulations as described in WO 2018/064472.

FIG. 5. shows the dissolution results for inventive composition 5425-68A at 0.1N HCl compared with placebo (i.e., no rifaximin) 5425-68B, Xifaxan 550 mg (Opadry II), and mg and 80 mg solid dispersion formulations as described in WO 2018/064472.

FIG. 6. shows the dissolution results for inventive composition 5425-70A, 5425-70B, and 5425-70C at pH 7.4 compared with placebo (i.e., no rifaximin) 5425-66B, Xifaxan 550 mg (Opadry II), and 40 mg and 80 mg solid dispersion formulations as described in WO 2018/064472.

FIG. 7. shows the dissolution results for inventive composition 5425-70A, 5425-70B, and 5425-70C at pH 4.5 compared with placebo (i.e., no rifaximin) 5425-66B, Xifaxan 550 mg (Opadry II), and 40 mg and 80 mg solid dispersion formulations as described in WO 2018/064472.

FIG. 8. shows the dissolution results for inventive composition 5425-70A, 5425-70B, and 5425-70C at 0.1N HCl compared with placebo (i.e., no rifaximin) 5425-66B, Xifaxan 550 mg (Opadry II), and 40 mg and 80 mg solid dispersion formulations as described in WO 2018/064472.

FIG. 9 shows the percent reduction, as a function of time (minutes), for E. Coli after treatment with inventive formulation 5507-65A compared to placebo and Xifaxan 550 mg.

FIG. 10 shows the percent reduction, as a function of time (minutes), for Salmonella choleraesuis after treatment with inventive formulation 5507-65A compared to placebo and Xifaxan 550 mg.

FIG. 11 shows the percent reduction, as a function of time (minutes), for Shigella flexneri after treatment with inventive formulation 5507-65A compared to placebo and Xifaxan 550 mg.

### DETAILED DESCRIPTION

Unless defined otherwise, all technical and scientific terms used herein have the same meaning as is commonly understood by a person having ordinary skill in the art to which this invention pertains.

When ranges are used herein to describe, for example, amounts of particular compounds or ingredients, all combinations and sub-combinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The term “comprising” (and related terms such as “comprise” or “comprises” or “having” or “including”) includes those embodiments such as, for example, an embodiment of any composition of matter, method or process that “consist of” or “consist essentially of” the described features.

### 1. Definitions

“Rifaximin” refers to the antibiotic 4-Deoxy-4′-methylpyrido[1′,2′-1,2]imidazo[5,4-c]rifamycin SV, having the chemical structure depicted in Formula I:

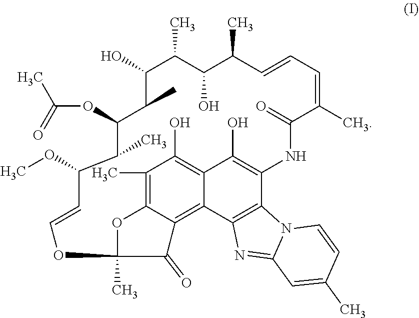 [SMILES: CO[C@H]1/C=C/O[C@@]2(C)Oc3c(C)c(O)c4c(O)c(c5c(nc6cc(C)ccn65)c4c3C2=O)NC(=O)/C(C)=C\C=C\[C@H](C)[C@H](O)[C@@H](C)[C@@H](O)[C@@H](C)[C@H](OC(C)=O)[C@@H]1C]

Forms, formulations, and methods of using rifaximin are described, for example, in U.S. Pat. Nos. 7,045,620, 7,906,542, 7,915,275, 8,193,196, 8,309,569, 8,518,949, 8,741,904, 9,737,610, the entirety of which are incorporated herein by reference.

As used herein the term “treatment of sickle cell disease (SCD)” refers to the amelioration, prevention, or reduction in frequency of one or more symptoms of SCD. For example, “treatment of sickle cell disease (SCD)” may refer to the reduction of elevated levels of circulating aged neutrophils (CANs) in a patient, where such levels of CANs are elevated as compared, for example, to levels of CANs that would be expected in a patient who is not diagnosed as having SCD. As another example, the “treatment of sickle cell disease (SCD)” may refer to treating vaso-occlusive crisis (VOC) in the patient, where “treating vaso-occlusive crisis (VOC) or “treating vaso-occlusive crises (VOCs),” as the case may be, may refer to (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. For example, symptoms of VOC include, without limitation, pain, swelling, ischemic injury, ulcers, priapism, organ damage, and spontaneous abortion. The “treatment of sickle cell disease (SCD)” may refer to preventing the occurrence of vaso-occlusive crisis (VOC) in a patient, such as a patient having a history of VOC (e.g., at least one VOC in the 12 months prior to treatment). The “treatment of sickle cell disease (SCD)” may refer to reducing the occurrence or frequency of vaso-occlusive crisis (VOC) in a patient, such as a patient having a history of VOC. The “treatment of sickle cell disease (SCD)” may refer to reducing the severity of vaso-occlusive crisis (VOC) occurrences in a patient, such as a patient having a history of VOC. The “treatment of sickle cell disease (SCD)” may refer to mediating or reducing a patient's opioid usage during VOC. The mediation or reduction of a patient's opioid usage during VOC may refer to, for example, mediating or reducing the patient's reliance on opioids (e.g., opioid analgesics) for pain management during VOC as compared to the patient's history of reliance on such opioids during VOC. Alternatively, the mediation or reduction of a patient's opioid usage during VOC may refer to mediating or reducing the patient's reliance on opioids (e.g., opioid analgesics) for pain management during VOC as compared to a patient during VOC who is not receiving a rifaximin composition as described herein.

The term “low dose rifaximin” means that rifaximin is present in an amount of 150 mg or less.

A “solubilizing excipient” refers to an inactive substance which is included in the disclosed compositions and which possess the ability to solubilize active pharmaceutical ingredients (APIs) such as rifaximin. Solubilizing excipients may be used, for example, e.g., in oral and injectable dosage forms. In one aspect, a “solubilizing excipient” refers to an excipient which provides for a rifaximin saturation solubility of greater than about 10% w/w. See, for example, the solubility pre-formulation procedure in the Exemplification section below. Solubilizing excipients include, but are not limited to, pH modifiers, organic solvents (e.g., water-soluble organic solvents), surfactants (e.g., non-ionic surfactants), lipids (e.g., water soluble lipids), long chain triglycerides, organic liquids/semi-solids), cyclodextrins, and phospholipids.

The term “solubilize” means to make soluble or to increase the solubility of a particular compound, such as rifaximin.

As used herein, an “antioxidant” refers to those substances which inhibit the oxidation of rifaximin, e.g., in a disclosed composition.

The term “effective amount” or “therapeutically effective amount” refers to an amount of a composition described herein that will elicit a biological or medical response of a subject, e.g., a composition having a dosage of rifaximin between about 0.001 to about 100 mg/kg body weight/day.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

“Pharmaceutically acceptable” means molecular entities and compositions that do not produce an adverse, allergic or other untoward reaction when administered to an animal, or a human, as appropriate.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

### 2. Compositions

Disclosed herein are pharmaceutical compositions in which the intestinal levels of soluble rifaximin are significantly enhanced as compared to Xifaxan® powder. See e.g., Table 8 where over a 150-fold increase in solubility was exhibited as compared to Xifaxan® powder. As shown herein, percent of soluble rifaximin available was observed in phosphate buffer at pH 7.4. Solubility increases using the disclosed compositions were also seen in intestinal fluid. See e.g., Tables 6A and 6B. Furthermore, the effect of antioxidant additives was explored and is demonstrated in Table 7.

The solubility results and further data described herein suggest that the disclosed compositions provide means for administering lower dosages of rifaximin without compromising therapeutic efficacy. Such compositions include, for example, pharmaceutically acceptable compositions comprising rifaximin (e.g., low dose rifaximin), a hydrogenated castor oil, and at least one additional solubilizing excipient. Formulations comprising one or more of the disclosed compositions, and their use in treating bowel related or liver function disorders are also provided.

### Rifaximin, a Hydrogenated Castor Oil, and at Least One Additional Solubilizing Excipient

In a first embodiment, provided herein are pharmaceutically acceptable compositions comprising rifaximin, a hydrogenated castor oil, and at least one additional solubilizing excipient. Alternatively, as part of a first embodiment, the rifaximin may be a low dose rifaximin.

In a second embodiment, the hydrogenated castor oil in the disclosed compositions, e.g., as in the first embodiment, is selected from polyoxyl 8 hydrogenated castor oil, polyoxyl 10 hydrogenated castor oil, polyoxyl 16 hydrogenated castor oil, polyoxyl 20 hydrogenated castor oil, polyoxyl 25 hydrogenated castor oil, polyoxyl 35 hydrogenated castor oil, polyoxyl 40 hydrogenated castor oil (e.g., Cremophor® RH-40), polyoxyl 45 hydrogenated castor oil, polyoxyl 50 hydrogenated castor oil, polyoxyl 54 hydrogenated castor oil, polyoxyl 55 hydrogenated castor oil, polyoxyl 60 hydrogenated castor oil, polyoxyl 65 hydrogenated castor oil, polyoxyl 80 hydrogenated castor oil, polyoxyl 100 hydrogenated castor oil, and polyoxyl 200 hydrogenated castor oil, and combinations thereof. Alternatively, as part of a second embodiment, the hydrogenated castor oil in the disclosed compositions, e.g., as in the first embodiment, is polyoxyl 60 hydrogenated castor oil or polyoxyl 40 hydrogenated castor oil. In another alternative, as part of a second embodiment, the hydrogenated castor oil in the disclosed compositions, e.g., as in the first embodiment, is polyoxyl 40 hydrogenated castor oil (e.g., Cremophor® RH-40).

In a third embodiment, the hydrogenated castor oil in the disclosed compositions, e.g., as in the first embodiment or second embodiment, is present in an amount ranging from about 25% to about 65%, about 25% to about 60%, about 25% to about 55%, about 25% to about 50%, about 30% to about 45%, about 35% to about 40%, about 30% to about 40%, about 31% to about 39%, about 32% to about 38%, about 33% to about 37%, about 34% to about 36%, about 40% to about 50%, about 41% to about 49%, about 42% to about 48%, about 43% to about 47%, or about 44% to about 46%; or present in an amount of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, or at least about 50%; or present in an amount of at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, or at most about 50%; or about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%; or present in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, by weight of the composition.

In an alternative third embodiment, the hydrogenated castor oil in the disclosed compositions, e.g., as in the first embodiment or second embodiment, is present in an amount ranging from about 25% to about 65%, about 25% to about 60%, about 25% to about 55%, about 25% to about 50%, about 30% to about 45%, about 35% to about 40%, about 30% to about 40%, about 31% to about 39%, about 32% to about 38%, about 33% to about 37%, about 34% to about 36%, about 40% to about 50%, about 41% to about 49%, about 42% to about 48%, about 43% to about 47%, or about 44% to about 46%; or present in an amount of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, or at least about 50%; or present in an amount of at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, or at most about 50%; or about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%; or present in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, by weight of the total weight of the rifaximin, hydrogenated castor oil, and at least one additional solubilizing excipient in the composition.

In a fourth embodiment, the at least one additional solubilizing excipient in the disclosed compositions, e.g., as in any one of the first through third embodiments, is selected from a water soluble organic solvent (e.g., polyethylene glycol (e.g., PEG 300, PEG 400, and PEG 600), ethanol, propylene glycol, benzyl alcohol, oleyl alcohol, triethylene glycol, n-methyl-2-pyrrolidone, dimethylacetamide, dimethylsulfoxide, diethylene glycol monoethyl ether (e.g., Transcutol® grades HP and P), diisopropyl adipate, and diethyl sebacate), a non-ionic surfactant (e.g., PEG-8 castor oil, PEG-9 castor oil, PEG-10 castor oil, PEG-11 castor oil, PEG-15 castor oil, PEG-16 castor oil, PEG-20 castor oil, PEG-25 castor oil, PEG-26 castor oil, PEG-29 castor oil, PEG-40 castor oil, PEG-44 castor oil, PEG-50 castor oil, PEG-54 castor oil, PEG-55 castor oil, PEG-60 castor oil, PEG-75 castor oil, PEG-80 castor oil, PEG-100 castor oil, PEG-200 castor oil, polyethylene glycol 1000 succinate, polysorbate 20, polysorbate 80, poly-oxyethylene esters of 12-hydroxystearic acid, sorbitan monooleate, poloxamer 407, oleoyl macrogol-6/polyoxyl-6 glycerides, caprylocaproyl macrogol-8/polyoxyl-8 glycerides, PEG-6 caprylic/capric glycerides, lauroyl polyoxyl-32 glycerides, and mono- and di-fatty acid esters of PEG 300, PEG 400, or PEG 1750), water-insoluble lipids (e.g., castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm oil), organic liquids/semi solids (e.g., beeswax, d-α-tocopherol, oleic acid, and medium chain mono-, di-, and triglycerides (e.g., glyceryl caprylate, glyceryl monooleate, glyceryl monolinoleate, and caprylic capric triglycerides)), cyclodextrins, (e.g., α-cyclodextrin, β-cyclodextrin, and hydroxypropyl-β-cyclodextrin), and phospholipids (e.g., hydrogenated soy phosphatidylcholine).

In a fifth embodiment, the at least one additional solubilizing excipient in the disclosed compositions, e.g., as in any one of the first through fourth embodiments, is selected from a water-soluble organic solvent, a non-ionic surfactant, a water-insoluble lipid, and long-chain triglycerides, and combinations thereof. Alternatively, as part of a fourth embodiment, the at least one additional solubilizing excipient in the disclosed compositions, e.g., as in any one of the first through fourth embodiments, is selected from polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, polysorbate 20, oleic acid, caprylic capric triglycerides, propylene glycol, sesame oil, soybean oil, and corn oil, and combinations thereof.

In a sixth embodiment, the at least one additional solubilizing excipient in the disclosed compositions, e.g., as in any one of the first through fifth embodiments, is one which allows for a rifaximin saturation solubility of greater than about 10% w/w (e.g., greater than about 11% w/w, greater than about 12% w/w, greater than about 13% w/w, greater than about 14% w/w, greater than about 15% w/w, greater than about 16% w/w, greater than about 17% w/w, greater than about 18% w/w, greater than about 19% w/w, or greater than about 20% w/w). Alternatively, as part of a sixth embodiment, the at least one additional solubilizing excipient in the disclosed compositions, e.g., as in any one of the first through fifth embodiments, is one which allows for a rifaximin saturation solubility of from about 10% w/w to about 25% w/w, from about 12% w/w to about 25% w/w, from about 12% w/w to about 23% w/w, from about 13% w/w to about 23% w/w, or from about 14% w/w to about 22% w/w.

In a seventh embodiment, the at least one additional solubilizing excipient in the disclosed compositions, e.g., as in any one of the first through sixth embodiments, is selected from polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, and combinations thereof. Alternatively, as part of a seventh embodiment, the at least one additional solubilizing excipient in the disclosed compositions, e.g., as in any one of the first through sixth embodiments, is selected from castor oil, glyceryl caprylate, polysorbate 80, diethyl sebacate, and diethylene glycol monoethyl ether, and combinations thereof.

In an eighth embodiment, the at least one additional solubilizing excipient in the disclosed compositions, e.g., as in any one of the first through seventh embodiments, is present in an amount ranging from about 25% to about 70%, about 25% to about 65%, about 30% to about 65%, about 35% to about 65%, about 40% to about 65%, about 40% to about 70%, about 45% to about 65%, about 46% to about 65%, about 47% to about 65%, about 48% to about 65%, about 49% to about 65%, about 50% to about 65%, about 45% to about 60%, about 46% to about 60%, about 47% to about 60%, about 48% to about 60%, about 49% to about 60%, about 50% to about 60%, about 51% to about 60%, about 52% to about 60%, about 53% to about 60%, about 54% to about 60%, or about 55% to about 60%; or present in an amount of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70%; or present in an amount of at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, or at most about 70%; or present in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%, by weight of the composition. Alternatively, as part of an eighth embodiment, the at least one additional solubilizing excipient in the disclosed compositions, e.g., as in any one of the first through seventh embodiments, is present in an amount ranging from about 51% to about 59%, about 52% to about 58%, about 53% to about 57%, or about 54% to about 56% by weight of the composition. Alternatively, as part of an eighth embodiment, the at least one additional solubilizing excipient in the disclosed compositions, e.g., as in any one of the first through seventh embodiments, is present in an amount ranging from about 59% to about 66%, about 60% to about 65%, about 61% to about 64%, or about 62% to about 63% by weight of the composition.

In an alternative eighth embodiment, the at least one additional solubilizing excipient in the disclosed compositions, e.g., as in any one of the first through seventh embodiments, is present in an amount ranging from about 40% to about 70%, about 45% to about 65%, about 46% to about 65%, about 47% to about 65%, about 48% to about 65%, about 49% to about 65%, about 50% to about 65%, about 45% to about 60%, about 46% to about 60%, about 47% to about 60%, about 48% to about 60%, about 49% to about 60%, about 50% to about 60%, about 51% to about 60%, about 52% to about 60%, about 53% to about 60%, about 54% to about 60%, about 55% to about 65%, about 62% to about 63%, or about 55% to about 60%; or present in an amount of at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70%; or present in an amount of at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, or at most about 70%; or present in an amount of about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%, by weight of the total weight of the rifaximin, hydrogenated castor oil, and at least one additional solubilizing excipient the composition. Alternatively, as part of an eighth embodiment, the at least one additional solubilizing excipient in the disclosed compositions, e.g., as in any one of the first through seventh embodiments, is present in an amount ranging from about 51% to about 59%, about 52% to about 58%, about 53% to about 57%, or about 54% to about 56% by weight of the total weight of the rifaximin, hydrogenated castor oil, and at least one additional solubilizing excipient in the composition.

In some embodiments of any one of the first through eighth embodiments, the hydrogenated castor oil and at least one additional solubilizing excipient may be provided in a ratio of about 1:3 and about 1.5:1, respectively, by weight hydrogenated castor oil and at least one additional solubilizing excipient. In some embodiments of any one of the first through eighth embodiments, the hydrogenated castor oil and at least one additional solubilizing excipient may be provided in a ratio of about 1:3 to about 1.5:1, respectively, by weight hydrogenated castor oil and at least one additional solubilizing excipient.

In some embodiments of any one of the first through eighth embodiments, the hydrogenated castor oil and at least one additional solubilizing excipient may be provided in a ratio of about 1:1 and about 1:2.5, respectively, by weight hydrogenated castor oil and at least one additional solubilizing excipient. In some embodiments of any one of the first through eighth embodiments, the hydrogenated castor oil and at least one additional solubilizing excipient may be provided in a ratio of about 1:1 to about 1:2.5, respectively, by weight hydrogenated castor oil and at least one additional solubilizing excipient.

### Rifaximin, a Hydrogenated Castor Oil, Castor Oil, Glyceryl Caprylate, and Polysorbate 80

In a ninth embodiment, the at least one additional solubilizing excipient in the disclosed compositions, e.g., as in any one of the first through eighth embodiments, is a combination of castor oil, glyceryl caprylate, and polysorbate 80.

In a tenth embodiment, the at least one additional solubilizing excipient in the disclosed compositions, e.g., as in any one of the first through ninth embodiments, is a combination of castor oil, glyceryl caprylate, and polysorbate 80, wherein:
the castor oil is present in an amount ranging from about 5% to about 15%, or about 6% to about 14%, or about 7% to about 13%, or about 8% to about 12%, or about 9% to about 11%; or present in an amount of at least about 5%, at least about 6%, at least about 7%, at least about 8%, at least about 9%, at least about 10%, at least about 11%, at least about 12%, at least about 13%, at least about 14%, or at least about 15%; or present in an amount of at most about 5%, at most about 6%, at most about 7%, at most about 8%, at most about 9%, at most about 10%, at most about 11%, at most about 12%, at most about 13%, at most about 14%, or at most about 15%; or present in an amount of about 5%, about 6%, about 7%, about 8%, about 9%, about 10%, about 11%, about 12%, about 13%, about 14%, or about 15%, by weight of the composition;
the glyceryl caprylate is present in an amount ranging from about 5% to about 15%, or about 10% to about 15%, or about 38% to about 42%, or about 6% to about 14%, or about 7% to about 13%, or about 8% to about 12%, or about 9% to about 11%; or present in an amount of at least about 5%, at least about 6%, at least about 7%, at least about 8%, at least about 9%, at least about 10%, at least about 11%, at least about 12%, at least about 13%, at least about 14%, or at least about 15%; or present in an amount of at most about 5%, at most about 6%, at most about 7%, at most about 8%, at most about 9%, at most about 10%, at most about 11%, at most about 12%, at most about 13%, at most about 14%, or at most about 15%; or present in an amount of about 5%, about 6%, about 7%, about 8%, about 9%, about 10%, about 11%, about 12%, about 13%, about 14%, or about 15%, by weight of the composition; and
the polysorbate 80 is present in an amount ranging from about 20% to about 40%, 35% to about 45%, or about 25% to about 40%, or about 27% to about 40%, or about 25% to about 37%, or about 27% to about 37%, or about 28% to about 36%, or about 30% to about 35%, or about 32% to about 35%; or present in an amount of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40%; or present in an amount of at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, or at most about 40%; or present in an amount of about 25%, about 26%, about 27%, about 28%, about 29%, about 30%, about 31%, about 32%, about 33%, about 34%, about 35%, about 36%, about 37%, about 38%, about 39%, or about 40%, by weight of the composition.

Alternatively, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 8% to about 12% by weight of the composition; the glyceryl caprylate is present in an amount ranging from about 10% to about 14% by weight of the composition; and the polysorbate 80 is present in an amount ranging from about 30% to about 35% by weight of the composition. In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein:
the castor oil is present in an amount ranging from about 10% to about 20% (e.g., about 11% to about 19%, about 12% to about 18%, about 13% to about 17%, or about 14% to about 16%), or present in an amount of at least about 10%, at least about 11%, at least about 12%, at least about 13%, at least about 14%, at least about 15%, at least about 16%, at least about 17%, at least about 18%, at least about 19%, or at least about 20%, or present in an amount of at most about 10%, at most about 11%, at most about 12%, at most about 13%, at most about 14%, at most about 15%, at most about 16%, at most about 17%, at most about 18%, at most about 19%, or at most about 20%, or present in an amount of about 10%, about 11%, about 12%, about 13%, about 14%, about 15%, about 16%, about 17%, about 18%, about 19%, or about 20%, by weight of the composition;
the glyceryl caprylate is present in an amount ranging from about 5% to about 15% (e.g., about 6% to about 14%, about 7% to about 13%, about 8% to about 12%, or about 9% to about 11%), or present in an amount of at least about 5%, at least about 6%, at least about 7%, at least about 8%, at least about 9%, at least about 10%, at least about 11%, at least about 12%, at least about 13%, at least about 14%, or at least about 15%, or present in an amount of at most about 5%, at most about 6%, at most about 7%, at most about 8%, at most about 9%, at most about 10%, at most about 11%, at most about 12%, at most about 13%, at most about 14%, or at most about 15%, or present in an amount of about 5%, about 6%, about 7%, about 8%, about 9%, about 10%, about 11%, about 12%, about 13%, about 14%, or about 15%, by weight of the composition; and
the polysorbate 80 is present in an amount ranging from about 20% to about 40% (e.g., about 25% to about 40%, about 27% to about 40%, about 25% to about 37%, about 27% to about 37%, about 28% to about 36%, about 30% to about 35%, or about 32% to about 35%), or present in an amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, or at most about 40%, or present in an amount of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, by weight of the composition.

In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 13% to about 18% by weight of the composition; the glyceryl caprylate is present in an amount ranging from about 10% to about 14% by weight of the composition; and the polysorbate 80 is present in an amount ranging from about 30% to about 35% by weight of the composition.

In an alternative tenth embodiment, the at least one additional solubilizing excipient in the disclosed compositions, e.g., as in any one of the first through ninth embodiments, is a combination of castor oil, glyceryl caprylate, and polysorbate 80, wherein:
the castor oil is present in an amount ranging from about 5% to about 15%, or about 6% to about 14%, or about 7% to about 13%, or about 8% to about 12%, or about 9% to about 11%; or present in an amount of at least about 5%, at least about 6%, at least about 7%, at least about 8%, at least about 9%, at least about 10%, at least about 11%, at least about 12%, at least about 13%, at least about 14%, or at least about 15%; or present in an amount of at most about 5%, at most about 6%, at most about 7%, at most about 8%, at most about 9%, at most about 10%, at most about 11%, at most about 12%, at most about 13%, at most about 14%, or at most about 15%; or present in an amount of about 5%, about 6%, about 7%, about 8%, about 9%, about 10%, about 11%, about 12%, about 13%, about 14%, or about 15%, by weight of the total weight of the rifaximin, hydrogenated castor oil, glyceryl caprylate, and polysorbate 80;
the glyceryl caprylate is present in an amount ranging from about 5% to about 15%, or about 6% to about 14%, or about 7% to about 13%, or about 8% to about 12%, or about 9% to about 11%; or present in an amount of at least about 5%, at least about 6%, at least about 7%, at least about 8%, at least about 9%, at least about 10%, at least about 11%, at least about 12%, at least about 13%, at least about 14%, or at least about 15%; or present in an amount of at most about 5%, at most about 6%, at most about 7%, at most about 8%, at most about 9%, at most about 10%, at most about 11%, at most about 12%, at most about 13%, at most about 14%, or at most about 15%; or present in an amount of about 5%, about 6%, about 7%, about 8%, about 9%, about 10%, about 11%, about 12%, about 13%, about 14%, or about 15%, by weight of the total weight of the rifaximin, hydrogenated castor oil, glyceryl caprylate, and polysorbate 80; and
the polysorbate 80 is present in an amount ranging from about 20% to about 40%, or about 25% to about 40%, or about 27% to about 40%, or about 25% to about 37%, or about 27% to about 37%, or about 28% to about 36%, or about 30% to about 35%, or about 32% to about 35%; or present in an amount of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40%; or present in an amount of at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, or at most about 40%; or present in an amount of about 25%, about 26%, about 27%, about 28%, about 29%, about 30%, about 31%, about 32%, about 33%, about 34%, about 35%, about 36%, about 37%, about 38%, about 39%, or about 40%, by weight of the total weight of the rifaximin, hydrogenated castor oil, glyceryl caprylate, and polysorbate 80.

Alternatively, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 8% to about 12% by weight of the total weight of the rifaximin, hydrogenated castor oil, glyceryl caprylate, and polysorbate 80; the glyceryl caprylate is present in an amount ranging from about 10% to about 14% by weight of the total weight of the rifaximin, hydrogenated castor oil, glyceryl caprylate, and polysorbate 80; and the polysorbate 80 is present in an amount ranging from about 30% to about 35% by weight of the total weight of the rifaximin, hydrogenated castor oil, glyceryl caprylate, and polysorbate 80. In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein:
the castor oil is present in an amount ranging from about 10% to about 20% (e.g., about 11% to about 19%, about 12% to about 18%, about 13% to about 17%, or about 14% to about 16%), or present in an amount of at least about 10%, 11%, 12%, 13%, 14%, 15%, 16%, 17%, 18%, 19%, or 20%, or present in an amount of at most about 10%, 11%, 12%, 13%, 14%, 15%, 16%, 17%, 18%, 19%, or 20%, or present in an amount of about 10%, 11%, 12%, 13%, 14%, 15%, 16%, 17%, 18%, 19%, or 20%, by weight of the total weight of the rifaximin, hydrogenated castor oil, glyceryl caprylate, and polysorbate 80;
the glyceryl caprylate is present in an amount ranging from about 5% to about 15% (e.g., about 6% to about 14%, about 7% to about 13%, about 8% to about 12%, or about 9% to about 11%), or present in an amount of at least about 5%, at least about 6%, at least about 7%, at least about 8%, at least about 9%, at least about 10%, at least about 11%, at least about 12%, at least about 13%, at least about 14%, or at least about 15%, or present in an amount of at most about at most about 5%, at most about 6%, at most about 7%, at most about 8%, at most about 9%, at most about 10%, at most about 11%, at most about 12%, at most about 13%, at most about 14%, or at most about 15%, or present in an amount of about 5%, about 6%, about 7%, about 8%, about 9%, about 10%, about 11%, about 12%, about 13%, about 14%, or about 15%, by weight of the total weight of the rifaximin, hydrogenated castor oil, glyceryl caprylate, and polysorbate 80; and
the polysorbate 80 is present in an amount ranging from about 20% to about 40% (e.g., about 25% to about 40%, about 27% to about 40%, about 25% to about 37%, about 27% to about 37%, about 28% to about 36%, about 30% to about 35%, or about 32% to about 35%), or present in an amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, or at most about 40%, or present in an amount of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, by weight of the total weight of the rifaximin, hydrogenated castor oil, glyceryl caprylate, and polysorbate 80.

In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 13% to about 18% by weight of the total weight of the rifaximin, hydrogenated castor oil, glyceryl caprylate, and polysorbate 80; the glyceryl caprylate is present in an amount ranging from about 10% to about 14% by weight of the composition; and the polysorbate 80 is present in an amount ranging from about 30% to about 35% by weight of the total weight of the rifaximin, hydrogenated castor oil, glyceryl caprylate, and polysorbate 80.

In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 5% to about 15% by weight of the composition; the glyceryl caprylate is present in an amount ranging from about 10% to about 15% by weight of the composition; and the polysorbate 80 is present in an amount ranging from about 35% to about 45% by weight of the composition.

In another alternative, as part of a tenth embodiment, the at least one additional solubilizing excipient in the disclosed compositions, e.g., as in any one of the first through ninth embodiments, is a combination of castor oil, glyceryl caprylate, and polysorbate 80, wherein the castor oil is present in an amount ranging from about 8% to about 12% by weight of the composition; the glyceryl caprylate is present in an amount ranging from about 10% to about 15% by weight of the composition; and the polysorbate 80 is present in an amount ranging from about 38% to about 42% by weight of the composition.

### Rifaximin, a Hydrogenated Castor Oil, Diethyl Sebacate, and Diethylene Glycol Monoethyl Ether

In an eleventh embodiment, the at least one additional solubilizing excipient in the disclosed compositions, e.g., as in any one of the first through eighth embodiments, is a combination of diethyl sebacate and diethylene glycol monoethyl ether. Alternatively, as part of an eleventh embodiment, the diethylene glycol monoethyl ether is diethylene glycol monoethyl ether.

In a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein:
the diethyl sebacate is present in an amount ranging from about 20% to about 35%, or about 21% to about 34%, or about 22% to about 33%, or about 23% to about 32%, or about 24% to about 31%, or about 25% to about 30%, or about 26% to about 29%, or about 26% to about 38%, or about 27% to about 28%, or present in amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, or at least about 35%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, or at most about 35%, or present in an amount of about 20%, about 21%, about 22%, about 23%, about 24%, about 25%, about 26%, about 27%, about 28%, about 29%, about 30%, about 31%, about 32%, about 33%, about 34%, or about 35%, by weight of the composition; and
the diethylene glycol monoethyl ether is present in an amount ranging from about 20% to about 35%, or about 21% to about 34%, or about 22% to about 33%, or about 23% to about 32%, or about 24% to about 31%, or about 25% to about 30%, or about 26% to about 29%, or about 26% to about 38%, or about 27% to about 28%, or present in amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, or at least about 35%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, or at most about 35%, or present in an amount of about 20%, about 21%, about 22%, about 23%, about 24%, about 25%, about 26%, about 27%, about 28%, about 29%, about 30%, about 31%, about 32%, about 33%, about 34%, or about 35%, by weight of the composition.

Alternatively, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein the diethyl sebacate is present in an amount ranging from about 25% to about 30% by weight of the composition; and the diethylene glycol monoethyl ether is present in an amount ranging from about 25% to about 30% by weight of the composition. In another alternative, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein:
the diethyl sebacate is present in an amount ranging from about 10% to about 20%, or about 11% to about 19%, or about 12% to about 18%, or about 13% to about 18%, or about 14% to about 18%, or about 15% to about 18%, or about 16% to about 18%, or about 16% to about 17%, or present in an amount of at least about 10%, at least about 11%, at least about 12%, at least about 13%, at least about 14%, at least about 15%, at least about 16%, at least about 17%, at least about 18%, at least about 19%, or at least about 20%, or present in an amount of at most about 10%, at most about 11%, at most about 12%, at most about 13%, at most about 14%, at most about 15%, at most about 16%, at most about 17%, at most about 18%, at most about 19%, or at most about 20%, or present in an amount of about 10%, about 11%, about 12%, about 13%, about 14%, about 15%, about 16%, about 17%, about 18%, about 19%, or about 20%, by weight of the composition; and
the diethylene glycol monoethyl ether is present in an amount ranging from about 30% to about 45%, or about 31% to about 44%, or about 32% to about 43%, or about 33% to about 42%, or about 34% to about 41%, or about 35% to about 40%, or about 36% to about 49%, or about 37% to about 49%, or present in an amount of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, or at least about 45%, or present in an amount of at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, or at most about 45%, or present in an amount of about 30%, about 31%, about 32%, about 33%, about 34%, about 35%, about 36%, about 37%, about 38%, about 39%, about 40%, about 41%, about 42%, about 43%, about 44%, or about 45%, by weight of the composition.

In another alternative, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein the diethyl sebacate is present in an amount ranging from about 14% to about 18% by weight of the composition; and the diethylene glycol monoethyl ether is present in an amount ranging from about 36% to about 40% by weight of the composition.

In an alternative twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein:
the diethyl sebacate is present in an amount ranging from about 20% to about 35%, or about 21% to about 34%, or about 22% to about 33%, or about 23% to about 32%, or about 24% to about 31%, or about 25% to about 30%, or about 26% to about 29%, or about 26% to about 38%, or about 27% to about 28%, or present in amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, or at least about 35%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, or at most about 35%, or present in an amount of about 20%, about 21%, about 22%, about 23%, about 24%, about 25%, about 26%, about 27%, about 28%, about 29%, about 30%, about 31%, about 32%, about 33%, about 34%, or about 35%, by weight of the total weight of the rifaximin, hydrogenated castor oil, diethyl sebacate, and diethylene glycol monoethyl ether; and
the diethylene glycol monoethyl ether is present in an amount ranging from about 20% to about 35%, or about 21% to about 34%, or about 22% to about 33%, or about 23% to about 32%, or about 24% to about 31%, or about 25% to about 30%, or about 26% to about 29%, or about 26% to about 38%, or about 27% to about 28%, or present in amount of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, or at least about 35%, or present in an amount of at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, or at most about 35%, or present in an amount of about 20%, about 21%, about 22%, about 23%, about 24%, about 25%, about 26%, about 27%, about 28%, about 29%, about 30%, about 31%, about 32%, about 33%, about 34%, or about 35%, by weight of the total weight of the rifaximin, hydrogenated castor oil, diethyl sebacate, and diethylene glycol monoethyl ether. Alternatively, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein the diethyl sebacate is present in an amount ranging from about 25% to about 30% by weight of the total weight of the rifaximin, hydrogenated castor oil, diethyl sebacate, and diethylene glycol monoethyl ether; and the diethylene glycol monoethyl ether is present in an amount ranging from about 25% to about 30% by weight of the total weight of the rifaximin, hydrogenated castor oil, diethyl sebacate, and diethylene glycol monoethyl ether. In another alternative, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein:
the diethyl sebacate is present in an amount ranging from about 10% to about 20%, or about 11% to about 19%, or about 12% to about 18%, or about 13% to about 18%, or about 14% to about 18%, or about 15% to about 18%, or about 16% to about 18%, or about 16% to about 17%, or present in an amount of at least about 10%, at least about 11%, at least about 12%, at least about 13%, at least about 14%, at least about 15%, at least about 16%, at least about 17%, at least about 18%, at least about 19%, or at least about 20%, or present in an amount of at most about 10%, at most about 11%, at most about 12%, at most about 13%, at most about 14%, at most about 15%, at most about 16%, at most about 17%, at most about 18%, at most about 19%, or at most about 20%, or present in an amount of about 10%, about 11%, about 12%, about 13%, about 14%, about 15%, about 16%, about 17%, about 18%, about 19%, or about 20%, by weight of the total weight of the rifaximin, hydrogenated castor oil, diethyl sebacate, and diethylene glycol monoethyl ether; and
the diethylene glycol monoethyl ether is present in an amount ranging from about 30% to about 45%, or about 31% to about 44%, or about 32% to about 43%, or about 33% to about 42%, or about 34% to about 41%, or about 35% to about 40%, or about 36% to about 49%, or about 37% to about 49%, or present in an amount of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, or at least about 45%, or present in an amount of at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, or at most about 45%, or present in an amount of about 30%, about 31%, about 32%, about 33%, about 34%, about 35%, about 36%, about 37%, about 38%, about 39%, about 40%, about 41%, about 42%, about 43%, about 44%, or about 45%, by weight of the total weight of the rifaximin, hydrogenated castor oil, diethyl sebacate, and diethylene glycol monoethyl ether.

In another alternative, as part of a twelfth embodiment, the at least one additional solubilizing excipient in the disclosed compositions, e.g., as in any one of the first through eighth and eleventh embodiments, is a combination of diethyl sebacate and diethylene glycol monoethyl ether, wherein the diethyl sebacate is present in an amount ranging from about 14% to about 18% by weight of the total weight of the rifaximin, hydrogenated castor oil, diethyl sebacate, and diethylene glycol monoethyl ether; and the diethylene glycol monoethyl ether is present in an amount ranging from about 36% to about 40% by weight of the total weight of the rifaximin, hydrogenated castor oil, diethyl sebacate, and diethylene glycol monoethyl ether.

### Antioxidants/Chelating Agents

In a thirteenth embodiment, the compositions described herein e.g., as in any one of the first through twelfth embodiments further comprise an antioxidant and/or a chelating agent.

In a fourteenth embodiment, the compositions described herein e.g., as in any one of the first through thirteenth embodiments further comprise an antioxidant and/or chelating agent selected from ascorbyl palmitate, butylated hydroxyanisole, butylated hydroxytoluene (BHT), citric acid, sodium metabisulfite, cysteine, potassium metabisulfite, propyl gallate, sodium thiosulfate, vitamin E, and 3,4-dihydroxybenzoic acid. Without being limited to any one theory of the invention, BHT and citric acid, for example, may be used as antioxidants and/or chelating agents to minimize potential degradation of rifaximin via oxidation.

Alternatively, as part of a fourteenth embodiment, the compositions described herein e.g., as in any one of the first through thirteenth embodiments further comprise BHT. In another alternative, as part of a fourteenth embodiment, the compositions described herein e.g., as in any one of the first through thirteenth embodiments further comprise BHT in an amount of less than about 1% (e.g., less than about 0.5%, less than about 0.1%, less than about 0.08%, less than about 0.06%, less than about 0.04%) by weight of the composition.

In another alternative, as part of a fourteenth embodiment, the compositions described herein e.g., as in any one of the first through thirteenth embodiments further comprise BHT in an amount ranging from about 0.01% to about 0.1%, from about 0.02% to about 0.08%, from about 0.025% to about 0.06%, from about 0.03% to about 0.06%, from about 0.03% to about 0.05%, from about 0.05% to about 0.15%, or from about 0.03% to about by weight of the composition.

Alternatively, as part of a fourteenth embodiment, the compositions described herein e.g., as in any one of the first through thirteenth embodiments further comprise citric acid. In another alternative, as part of a fourteenth embodiment, the compositions described herein e.g., as in any one of the first through thirteenth embodiments further comprise citric acid in an amount of less than about 1% (e.g., less than about 0.5%, less than about 0.1%, less than about 0.08%, less than about 0.06%, less than about 0.04%) by weight of the composition.

In another alternative, as part of a fourteenth embodiment, the compositions described herein e.g., as in any one of the first through thirteenth embodiments further comprise citric acid in an amount ranging from about 0.01% to about 0.1%, from about to about 0.08%, from about 0.025% to about 0.06%, from about 0.03% to about from about 0.03% to about 0.05%, or from about 0.03% to about 0.04% by weight of the composition.

Alternatively, as part of a fourteenth embodiment, the compositions described herein e.g., as in any one of the first through thirteenth embodiments further comprise ascorbyl palmitate. Alternatively, as part of a fourteenth embodiment, the compositions described herein e.g., as in any one of the first through thirteenth embodiments further comprise ascorbyl palmitate in an amount ranging from about 0.05% to about 0.15%, from about 0.06% to about 0.14%, from about 0.07% to about 0.13%, from about 0.08% to about or from about 0.08% to about 0.12% by weight of the composition.

Alternatively, as part of a fourteenth embodiment, the compositions described herein e.g., as in any one of the first through thirteenth embodiments further comprise BHT and citric acid. In another alternative, as part of a fourteenth embodiment, the compositions described herein e.g., as in any one of the first through thirteenth embodiments further comprise BHT and citric acid each in an amount of less than about 1% (e.g., less than about less than about 0.1%, less than about 0.08%, less than about 0.06%, less than about by weight of the composition.

In another alternative, as part of a fourteenth embodiment, the compositions described herein e.g., as in any one of the first through thirteenth embodiments further comprise BHT and citric acid each in an amount ranging from about 0.01% to about 0.1%, from about 0.02% to about 0.08%, from about 0.025% to about 0.06%, from about 0.03% to about 0.06%, from about 0.03% to about 0.05%, or from about 0.03% to about 0.04% by weight of the composition.

In another alternative, as part of a fourteenth embodiment, the compositions described herein e.g., as in any one of the first through thirteenth embodiments further comprise BHT, citric acid, and ascorbyl palmitate each in an amount ranging from about to about 0.15%, from about 0.06% to about 0.14%, from about 0.7% to about 0.13%, from about 0.8% to about 0.12%, or from about 0.09% to about 0.11%, by weight of the composition.

### Rifaximin

In a fifteenth embodiment, the rifaximin in the disclosed compositions, e.g., as in any one of the first through fourteenth embodiments, is present in an amount ranging from about 1% to about 15%, about 1% to about 5%, or about 2.5% to about 15%, or about 2.5% to about 12%, or about 5% to about 10%, or about 7% to about 15%, or about 7% to about 14%, or about 8% to about 14%, or about 8% to about 13%, or about 8% to about 12%, or about 8% to about 11%, or about 9% to about 12%, or about 9% to about 12%, or about 1% to about 12%, or 1% to about 10%, or about 1% to about 8%, or 2% to about 7%, 3% to about 7%, or 3% to about 6%, or 4% to about 5%, or present in an amount of at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, or at least about 15%, or present in an amount of at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, or at most about 15%, or present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, or about 15%, by weight of the composition.

In an alternative fifteenth embodiment, the rifaximin in the disclosed compositions, e.g., as in any one of the first through fourteenth embodiments, is present in an amount ranging from about 1% to about 15%, or about 2.5% to about 15%, or about 2.5% to about 12%, or about 5% to about 10%, or about 7% to about 15%, or about 7% to about 14%, or about 8% to about 14%, or about 8% to about 13%, or about 8% to about 12%, or about 8% to about 11%, or about 9% to about 12%, or about 9% to about 12%, or about 1% to about 12%, or 1% to about 10%, or about 1% to about 8%, or 2% to about 7%, 3% to about 7%, or 3% to about 6%, or 4% to about 5%, or present in an amount of at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, or at least about 15%, or present in an amount of at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, or at most about 15%, or present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, or about 15%, by weight of the rifaximin, hydrogenated castor oil, and at least one additional solubilizing excipient.

In a sixteenth embodiment, the total amount of rifaximin in the disclosed composition, e.g., as in any one of the first through fifteenth embodiments, is less than about 125 mg (e.g., less than about 120 mg, less than about 110 mg, less than about 100 mg, less than about 90 mg, less than about 80 mg, less than about 70 mg, less than about 60 mg, less than about 50 mg, less than about 40 mg, less than about 30 mg, less than about 20 mg, or less than about 15 mg, less than about 10 mg, or less than about 9 mg, or less than about 8 mg, or less than about 7 mg, or less than about 6 mg, or less than about 5 mg, or less than about 4 mg, or less than about 3 mg, or less than about 2 mg, or less than about 1 mg, or less than about 0.5 mg). Alternatively, as part of a sixteenth embodiment, the total amount of rifaximin in the disclosed composition, e.g., as in any one of the first through fifteenth embodiments, ranges from about 1 mg to about 125 mg (e.g., about 1 mg to about 5 mg, about 2 mg to about 5 mg, about 3 mg to about 5 mg, about 3 mg to about 4 mg, about 1 mg to about 125 mg, about 5 mg to about 125 mg, about 10 mg to about 125 mg, about 10 mg to about 100 mg, about 25 mg to about 125 mg, about 25 mg to about 100 mg, about 25 mg to about 75 mg, about 30 mg to about 70 mg, about 35 mg to about 65 mg, about 40 mg to about 60 mg, about 45 mg to about 55 mg, about 75 mg to about 125 mg, about 80 mg to about 120 mg, 85 mg to about 115 mg, about 90 mg to about 110 mg, or about 95 mg to about 105 mg). Alternatively, as part of a sixteenth embodiment, the total amount (in milligrams) of rifaximin in the disclosed composition, e.g., as in any one of the first through fifteenth embodiments, is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 mg; or at least about 1, at least about 1.5, at least about 2, at least about 2.5, at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, at least about 10, at least about 10.5, at least about 11, at least about 11.5, at least about 12, at least about 12.5, at least about 13, at least about 13.5, at least about 14, at least about 14.5, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, or at least about 125 mg; or is at most about 1, at most about 1.5, at most about 2, at most about 2.5, at most about 3, at most about 3.5, at most about 4, at most about 4.5, at most about 5, at most about 5.5, at most about 6, at most about 6.5, at most about 7, at most about 7.5, at most about 8, at most about 8.5, at most about 9, at most about 9.5, at most about 10, at most about 10.5, at most about 11, at most about 11.5, at most about 12, at most about 12.5, at most about 13, at most about 13.5, at most about 14, at most about 14.5, at most about 15, at most about 16, at most about 17, at most about 18, at most about 19, at most about 20, at most about 21, at most about 22, at most about 23, at most about 24, at most about 25, at most about 26, at most about 27, at most about 28, at most about 29, at most about 30, at most about 31, at most about 32, at most about 33, at most about 34, at most about 35, at most about 36, at most about 37, at most about 38, at most about 39, at most about 40, at most about 41, at most about 42, at most about 43, at most about 44, at most about 45, at most about 46, at most about 47, at most about 48, at most about 49, at most about 50, at most about 51, at most about 52, at most about 53, at most about 54, at most about 55, at most about 56, at most about 57, at most about 58, at most about 59, at most about 60, at most about 61, at most about 62, at most about 63, at most about 64, at most about 65, at most about 66, at most about 67, at most about 68, at most about 69, at most about 70, at most about 71, at most about 72, at most about 73, at most about 74, at most about 75, at most about 76, at most about 77, at most about 78, at most about 79, at most about 80, at most about 81, at most about 82, at most about 83, at most about 84, at most about 85, at most about 86, at most about 87, at most about 88, at most about 89, at most about 90, at most about 91, at most about 92, at most about 93, at most about 94, at most about 95, at most about 96, at most about 97, at most about 98, at most about 99, at most about 100, at most about 101, at most about 102, at most about 103, at most about 104, at most about 105, at most about 106, at most about 107, at most about 108, at most about 109, at most about 110, at most about 111, at most about 112, at most about 113, at most about 114, at most about 115, at most about 116, at most about 117, at most about 118, at most about 119, at most about 120, at most about 121, at most about 122, at most about 123, at most about 124, or at most about 125 mg.

Alternatively, as part of an additional embodiment, the total amount (in milligrams) of rifaximin in the disclosed composition, e.g., as in any one of the first through sixteenth embodiments, is at least about 0.5, at least about 1.0, at least about 1.5, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, at least about 10.0, at least about 10.5, or at least about 11 mg, or at most about at most about 1.0, at most about 1.5, at most about 2.0, at most about 2.5, at most about 3.0, at most about 3.5, at most about 4.0, at most about 4.5, at most about 5.0, at most about at most about 6.0, at most about 6.5, at most about 7.0, at most about 7.5, at most about 8.0, at most about 8.5, at most about 9.0, at most about 9.5, at most about 10.0, at most about or at most about 11 mg, or about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, or about 11 mg.

### Liquid Composition

In a seventeenth embodiment, the compositions described herein, e.g., as in any one of the first through sixteenth embodiments, is a liquid composition.

### 3. Dosage Forms

For the purposes of administration, in certain embodiments, the compositions described herein may be administered as is or formulated as alternative dosage forms, e.g., for orally delivery. Formulations for oral delivery can be in the form of lozenges, aqueous or oily suspensions, emulsions, capsules, syrups, or elixirs. Orally administered compositions can comprise one or more optional agents, for example, sweetening agents such as fructose, aspartame or saccharin; flavoring agents such as peppermint, oil of wintergreen, or cherry; coloring agents; and preserving agents, to provide a pharmaceutically-palatable preparation. The compositions may be coated to delay disintegration and absorption in the gastrointestinal tract thereby providing a sustained action over an extended period of time.

In certain embodiments, the disclosed compositions are formulated in capsule dosage forms. In certain embodiments, the disclosed compositions are formulated in soft or hard capsule dosage forms. In certain embodiments, the disclosed compositions are formulated in soft or hard gelatin capsule dosage forms.

It should be noted that a specific dosage and treatment regimen for any particular patient will depend upon a variety of factors, including age, body weight, general health, sex, diet, time of administration, rate of excretion, drug combination, the judgment of the treating physician, and the severity of the particular disease being treated.

### 4. Methods of Use

The compositions described herein may be used in methods for treating sickle cell disease (SCD). In some embodiments, the method of treating sickle cell disease (SCD) comprises reducing elevated levels of circulating aged neutrophils (CANs) in the patient. In some embodiments, the method of treating sickle cell disease (SCD) in a patient comprises treating vaso-occlusive crisis (VOC) in the patient.

In some embodiments, treating vaso-occlusive crisis (VOC) in the patient comprises (1) alleviating one or more symptoms of VOC in the patient; (2) reducing or preventing the occurrence of VOCs in the patient; (3) reducing the duration or severity of VOC in the patient; and/or (4) mediating or otherwise reducing the patient's opioid usage during VOC. In some embodiments, the method of treating sickle cell (SCD) in the patient comprises alleviating one or more symptoms of vaso-occlusive crisis (VOC) in the patient. In some embodiments, the method of treating sickle cell disease (SCD) in the patient comprises reducing or preventing the occurrence of vaso-occlusive crises (VOCs) in the patient. In some embodiments, the method of treating sickle cell disease (SCD) in the patient comprises reducing the duration or severity of VOC in the patient. In some embodiments, the method of treating sickle cell disease (SCD) in the patient comprises mediating or otherwise reducing the patient's opioid usage during vaso-occlusive crisis (VOC) in the patient.

In some embodiments, the methods described herein may include administering an aforementioned composition QD, BID, TID, or QID to a subject to provide a daily dose (in milligrams) of rifaximin to the subject in an amount of at least about 1.0, at least about 1.5, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, at least about 10.0, at least about 10.5, at least about 11.0, at least about 11.5, at least about 12.0, at least about 12.5, at least about 13.0, at least about 13.5, at least about 14.0, at least about 14.5, at least about 15.0, at least about 15.5, at least about 16.0, at least about 16.5, at least about 17.0, at least about 17.5, at least about 18.0, at least about 18.5, at least about 19.0, at least about 19.5, at least about 20.0, at least about at least about 21.0, at least about 21.5, at least about 22.0, at least about 22.5, at least about 23.0, at least about 23.5, at least about 24.0, at least about 24.5, at least about 25.0, at least about 25.5, at least about 26.0, 26.5, at least about 27.0, at least about 27.5, at least about 28.0, at least about 28.5, at least about 29.0, at least about 29.5, at least about 30.0, at least about 30.5, at least about 31.0, at least about 31.5, at least about 32.0, at least about 32.5, at least about 33.0, at least about 33.5, at least about 34.0, at least about 34.5, at least about 35.0, at least about 35.5, at least about 36.0, at least about 36.5, at least about 37.0, at least about 37.5, at least about 38.0, at least about 38.5, at least about 39.0, at least about 39.5, or at least about 40.0 mg. In some embodiments, the methods described herein may include administering an aforementioned composition QD, BID, TID, or QID to a subject to provide a daily dose of rifaximin to the subject in an amount (in milligrams) of at most about 1.0, at most about 1.5, at most about 2.0, at most about 2.5, at most about 3.0, at most about 3.5, at most about 4.0, at most about 4.5, at most about 5.0, at most about 5.5, at most about 6.0, at most about 6.5, at most about 7.0, at most about 7.5, at most about 8.0, at most about 8.5, at most about 9.0, at most about 9.5, at most about 10.0, at most about 10.5, at most about 11.0, at most about 11.5, at most about 12.0, at most about 12.5, at most about 13.0, at most about 13.5, at most about 14.0, at most about 14.5, at most about 15.0, at most about at most about 16.0, at most about 16.5, at most about 17.0, at most about 17.5, at most about 18.0, at most about 18.5, at most about 19.0, at most about 19.5, at most about 20.0, at most about 20.5, at most about 21.0, at most about 21.5, at most about 22.0, at most about 22.5, at most about 23.0, at most about 23.5, at most about 24.0, at most about 24.5, at most about 25.0, at most about 25.5, at most about 26.0, 26.5, at most about 27.0, at most about 27.5, at most about 28.0, at most about 28.5, at most about 29.0, at most about 29.5, at most about 30.0, at most about 30.5, at most about 31.0, at most about 31.5, at most about 32.0, at most about 32.5, at most about 33.0, at most about 33.5, at most about 34.0, at most about 34.5, at most about 35.0, at most about 35.5, at most about 36.0, at most about 36.5, at most about 37.0, at most about 37.5, at most about 38.0, at most about 38.5, at most about 39.0, at most about 39.5, or at most about 40.0 mg. In some embodiments, the methods described herein may include administering an aforementioned composition QD, BID, TID, or QID to a subject to provide a daily dose of rifaximin to the subject in an amount (in milligrams) of about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, about 21.0, about 21.5, about 22.0, about 22.5, about 23.0, about 23.5, about 24.0, about 24.5, about 25.0, about 25.5, about 26.0, 26.5, about 27.0, about 27.5, about 28.0, about 28.5, about 29.0, about 29.5, about 30.0, about 30.5, about 31.0, about 31.5, about 32.0, about 32.5, about 33.0, about 33.5, about 34.0, about 34.5, about 35.0, about 35.5, about 36.0, about 36.5, about 37.0, about 37.5, about 38.0, about 38.5, about 39.0, about 39.5, or about 40.0 mg.

In some embodiments, the methods described herein may include administering an aforementioned composition at a dose of 3.5 mg, or 7.0 mg, or 10.5 mg BID to provide a daily dose of rifaximin to the subject in an amount of about 7.0 mg, or 14.0 mg, or 21.0 mg, respectively.

In some embodiments, the foregoing doses or daily doses may be provided by administering at least 1, 2, 3, 4, 5, 6, 7, 8, 9, or 10 unit dosage forms of the composition (e.g., capsules) to a subject per dose or per day, as the case may be. In some embodiments, the foregoing doses or daily doses may be provided by administering at most 1, 2, 3, 4, 5, 6, 7, 8, 9, or 10 unit dosage forms of the composition (e.g., capsules) to a subject per dose or per day, as the case may be. In some embodiments, the foregoing doses or daily doses may be provided by administering about 1, 2, 3, 4, 5, 6, 7, 8, 9, or 10 unit dosage forms of the composition (e.g., capsules) to a subject per dose or per day, as the case may be.

In some embodiments, the rifaximin compositions described herein may be administered with an additional SCD therapeutic agent in the foregoing methods of treatment. In some embodiments, the additional SCD therapeutic agent may be, for example, hydroxyurea, L-glutamine, hydroxycarbamide, an erythropoietin stimulating agent, and/or an opioid analgesic. In some embodiments, the opioid analgesic may be selected from the group consisting of morphine, codeine, hydrocodone, hydromorphone, methadone, tramadol, oxycodone, tapentadol, fentanyl, and a combination thereof.

While certain embodiments of the invention have been described and/or exemplified above, various other embodiments will be apparent to those skilled in the art from this disclosure. The invention is, therefore, not limited to the particular embodiments described and/or exemplified, but is capable of considerable variation and modification without departure from the scope and spirit of the appended claims.

### Exemplification

The representative examples that follow are intended to help illustrate the present disclosure, and are not intended to, nor should they be construed to, limit the scope of the invention.

### 1. Pre-Formulation Studies

A. Solubility

The solubility of rifaximin in various solubilizing excipients was investigated by adding excess amount of rifaximin in each solubilizing excipient to determine the saturation solubility. The amount of rifaximin dissolved was analyzed by HPLC. The results from the solubility screen are shown below in Table 1.

TABLE 1

| Solubilizing | Solubility | Solubilizing | Solubility |
|---|---|---|---|
| Excipient | (% w/w) | Excipient | (% w/w) |
| Diethylene glycol | 21.25 | Glyceryl caprylate | 20.20 |
| monoethyl ether grade | | | |
| HP | | | |
| Polyoxyl 40 | 20.30 | Castor oil | 17.10 |
| hydrogenated | | | |
| castor oil | | | |
| Polysorbate 80 | 19.90 | Glyceryl monooleate | 15.30 |
| | | (Type 40) | |
| Benzyl alcohol | 16.22 | Glyceryl | 15.00 |
| | | monolinoleate | |
| Polyethylene | 15.12 | Triethylene glycol | 14.62 |
| glycol 400 | | | |
| Diethyl sebacate | 11.22 | Oleyl alcohol | 10.27 |
| Polysorbate 20 | 9.27 | Oleic acid | 3.79 |
| Caprylic capric | 2.00 | Propylene glycol | 0.73 |
| triglycerides | | | |
| Sesame oil | 0.60 | Soybean oil | 0.55 |
| Corn oil | 0.30 | Diisopropyl adipate | 12.71 |
| Polyethylene glycol 600 | 21.20 | | |

B. Excipient Compatibility

To determine excipient compatibility, pre-determined amounts of rifaximin were added to each solvent and the resulting solutions were subjected to various temperature conditions for up to 4 weeks. Chemical purity was then analyzed via HPLC, the results of which are shown in Table 2.

TABLE 2

| | | Rifaximin % Label Claim (LC) | | | | | |
|---|---|---|---|---|---|---|---|
| | | T 2 weeks | | | T 4 weeks | | |
| | | 25° | 40° | 50° | 25° | 40° | 50° |
| Solvent | T0 | C. | C. | C. | C. | C. | C. |
| Castor Oil | 94.0 | 99.3 | 104.3 | 106.4 | 100.7 | 104.3 | 101.4 |
| Diethyl | 102.7 | 102.1 | 101.7 | 101.7 | 101.1 | 98.8 | 99.8 |
| sebacate | | | | | | | |
| Propylene | 97.9 | 96.9 | 97.0 | 95.7 | 96.2 | 95.6 | 98.6 |
| glycol | | | | | | | |
| Polyoxyl 40 | 102.7 | 100.0 | 100.0 | 97.4 | 101.3 | 99.4 | 96.1 |
| hydrogenated | | | | | | | |
| castor oil | | | | | | | |
| PEG 600 | 102.0 | 98.0 | 98.7 | 98.0 | 100.0 | 99.3 | 96.1 |
| Polysorbate | 104.7 | 98.7 | 98.1 | 96.8 | 96.8 | 96.8 | 94.3 |
| 80 | | | | | | | |
| Benzyl | 99.3 | 99.8 | 96.3 | 96.9 | 98.2 | 94.0 | 93.0 |
| alcohol | | | | | | | |
| Glyceryl | 102.7 | 98.7 | 100.0 | 97.4 | 100.6 | 100.0 | 92.9 |
| monolinoleate | | | | | | | |
| Triethylene | 97.5 | 98.3 | 96.7 | 95.6 | 97.0 | 95.0 | 92.5 |
| glycol | | | | | | | |
| Oleyl alcohol | 98.7 | 96.3 | 97.1 | 95.2 | 96.9 | 95.0 | 91.8 |
| Caprylic/ | 86.8 | 92.2 | 93.0 | 94.5 | NT | 92.0 | 89.3 |
| capric | | | | | | | |
| triglyceride | | | | | | | |
| PEG 400 | 98.1 | 96.0 | 95.4 | 91.8 | 95.8 | 92.2 | 86.7 |
| diethylene | 97.3 | 96.1 | 89.3 | 81.5 | 95.4 | 82.7 | 74.2 |
| glycol | | | | | | | |
| monoethyl | | | | | | | |
| ether grade P | | | | | | | |
| Glyceryl | 92.7 | 96.4 | 58.3 | 48.9 | 95.0 | 59.0 | 41.7 |
| monooleate | | | | | | | |
| Diisopropyl | 97.6 | 95.3 | 71.3 | 50.3 | 95.1 | 64.8 | 30.4 |
| adipate | | | | | | | |

C. Compatibility with Gelatin Shells

The physical compatibility of individual excipients with gelatin shell matrix was investigated. For hard gelatin capsules, solvent was filled into hard capsules and placed in scintillation vials. For soft gelatin capsules, each capsule was dipped into the excipient and then placed in scintillation vials. The vials were then subjected to various temperature conditions for up to 4 weeks. Capsules were then evaluated for physical changes (softening or hardening of capsule shell).

Hard gel capsules were purchased from VWR (catalogue number 70102) with a volume of 0.68 mL, diameter of 6.63 mm, a length of 19.0 mm and size 1. Soft gel capsules were Advil liquid-gels, 200 mg strength, 80 counts. Results are shown in Tables 3 and 4.

TABLE 3
Hard Gel Capsules

| | Physical change (Yes or No) | | | | | |
|---|---|---|---|---|---|---|
| | T 2 weeks | | | T 4 weeks | | |
| Solvent | 25° C. | 40° C. | 50° C. | 25° C. | 40° C. | 50° C. |
| PEG 600 | No | No | No | No | No | No |
| PEG 400 | No | Yes | — | No | Yes | — |
| Polysorbate | No | No | No | No | No | No |
| 80 | | | | | | |
| Castor oil | No | No | No | No | No | No |
| Polyoxyl 40 | No | No | No | No | No | No |
| hydrogenated | | | | | | |
| castor oil | | | | | | |
| Glyceryl | No | No | No | No | No | No |
| monooleate | | | | | | |
| Glyceryl | No | No | No | No | No | No |
| monolinoleate | | | | | | |
| Corn oil | No | No | No | No | No | No |
| diethylene | No | No | No | No | No | No |
| glycol | | | | | | |
| monoethyl | | | | | | |
| ether grade | | | | | | |
| HP | | | | | | |
| Diethyl | No | No | — | No | No | — |
| sebacate | | | | | | |
| Benzyl | Yes | Yes | — | Yes | Yes | — |
| alcohol | | | | | | |
| Triethylene | Yes | Yes | — | Yes | Yes | — |
| glycol | | | | | | |
| Oleyl alcohol | No | No | — | No | No | — |
| Caprylic/ | No | No | — | No | No | — |
| capric | | | | | | |
| triglyceride | | | | | | |

TABLE 4
Soft Gel Capsules

| | Physical Change (Yes or No) | | | | | |
|---|---|---|---|---|---|---|
| | T 2 weeks | | | T 4 weeks | | |
| Solvent | 25° C. | 40° C. | 50° C. | 25° C. | 40° C. | 50° C. |
| PEG 600 | No | No | No | No | No | No |
| Polysorbate | No | No | No | No | No | No |
| 80 | | | | | | |
| Castor oil | No | No | No | No | No | No |
| Polyoxyl 40 | No | No | Yes | No | No | Yes |
| Hydrogenated | | | | | | |
| castor oil | | | | | | |
| PEG 400 | No | No | No | No | No | No |
| Glyceryl | No | Yes | Yes | No | Yes | Yes |
| monooleate | | | | | | |
| Glyceryl | No | No | No | No | No | No |
| monolinoleate | | | | | | |

### 2. Formulation Studies

A. Evaluation of Percent of Soluble Drug Available

The percent of drug (rifaximin) soluble in phosphate buffer (pH˜6.8) or simulated intestinal fluid (SIF, pH˜6.8) was investigated using the following criteria. The control species was Xifaxan® tablet (200 mg) or Xifaxan® powder (200 mg API+glyceryl distearate—to represent enteric coated tablet.) Additives, where noted, included bile acid (cholic acid), butylated hydroxytoluene (BHT), and isooctyl acrylate/acrylamide/vinyl acetate copolymer (Kollidon® VA64).

Compositions were made by weighing the hydrogenated castor oil and at least one additional solubilizing excipient followed by mixing. Where applicable, BHT was then added to the mixture and dissolved. Rifaximin was added last.

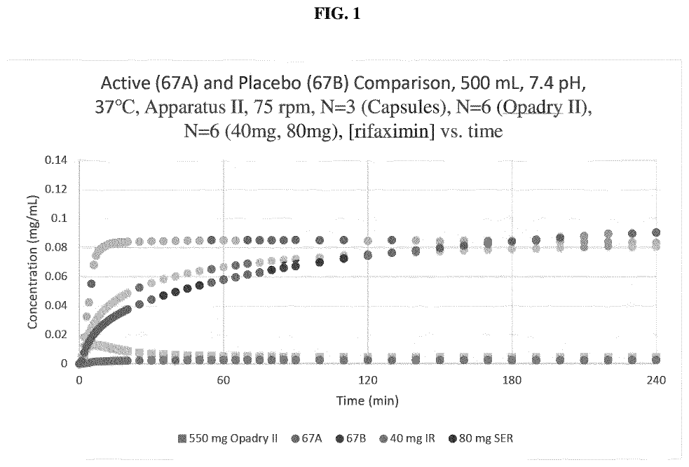

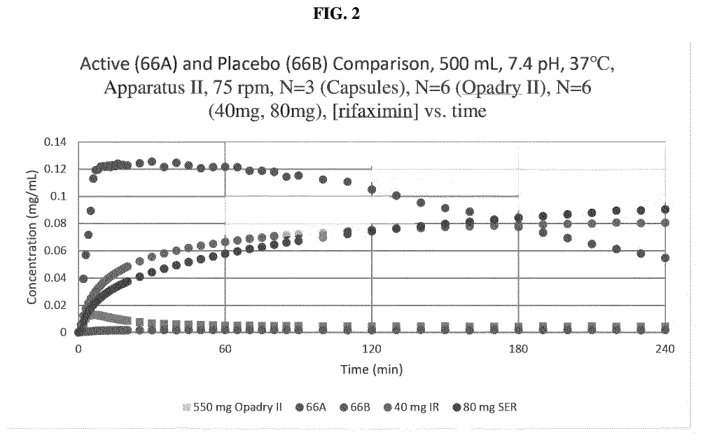

Dissolution studies were performed to evaluate the percent drug solubilized over a period of time from Rifaximin SEDDS formulations as compared to Xifaxan at pH 7.4 buffer, 37 degrees Celsius. Samples were analyzed via HPLC. Results are shown below in Table 5 as well as FIG. 1 and FIG. 2. As shown in the figures, dissolution of the inventive formulations (5425-66A; 70 mg rifaximin and 5425-67A; 35 mg rifaximin) was significantly faster than previously disclosed 40 mg rifaximin IR and 80 mg rifaximin SER solid dispersion compositions (see WO 2018/064472) and Xifaxan 550 mg.

TABLE 5

% w/w

| Batch No. | 5425-66A | 5425-67A |
|---|---|---|
| API concentration | 10% | 5% |
| | API | API |
| Rifaximin | 10.00 | 5.00 |
| Castor Oil | 10.00 | 15.00 |
| Polyoxyl 40 Hydrogenated Castor Oil | 34.96 | 34.96 |
| Glyceryl Caprylate Type I | 12.00 | 12.00 |
| Polysorbate (Tween) 80 | 33.00 | 33.00 |
| Butylated Hydroxytoluene (BHT) | 0.04 | 0.04 |
| Total | 100.00 | 100.00 |
| Ratio of % Drug soluble as | 71.59 | 35.42 |
| compared to Xifaxan at 1 hour, pH | | |
| 7.4 buffer | | |

For SIF, prototype compositions (1 gram total, each comprising 100 mg of API) or Xifaxan® powder were mixed at 37° C. for 1 hour in SIF (100 mL). The mixture was then centrifuged for 20 min and analyzed via HPLC. Results are shown below in Table 6A and 6B.

TABLE 6A
Percent Soluble Drug in Phosphate Buffer

| | 5256- | 5394- | 5256- | 5394- | 5256- | 5394- | Xifaxan ® |
|---|---|---|---|---|---|---|---|
| | 27C | 58A | 34E | 57A | 35A | 60A | powder |
| Ingredient | w/w % | | | | | | |
| Rifaximin | 10.0 | 5.00 | 10.0 | 5.00 | 10.0 | 5.00 | 200 mg |
| Castor oil | 10.0 | 15.00 | — | — | — | — | — |
| Glyceryl | 12.0 | 12.00 | — | — | — | — | — |
| caprylate | | | | | | | |
| Polysorbate | 33.0 | 33.0 | — | — | — | — | — |
| (Tween) 80 | | | | | | | |
| Polyoxyl 40 | 34.96 | 34.96 | 35.0 | 19.96 | 34.96 | 39.96 | — |
| Hydrogenated | | | | | | | |
| Castor | | | | | | | |
| Diethyl Sebacate | — | — | 27.5 | 37.50 | 27.5 | 27.50 | — |
| Diethylene | — | — | 27.5 | 37.50 | 27.5 | 27.50 | |
| glycol | | | | | | | |
| monoethyl ether | | | | | | | |
| Butylated | 0.038 | 0.038 | — | 0.038 | 0.038 | 0.038 | — |
| hydroxytoluene | | | | | | | |
| Glyceryl | — | — | — | — | — | — | 18 mg |
| distearate | | | | | | | |
| Total | 100.00 | 100.00 | 100.00 | 100.00 | 100.00 | 100.00 | 218.00 |
| Percent of drug | 0.0131 | 0.0503 | 0.0021 | 0.0064 | 0.0024 | 0.0065 | 0.0003 |
| soluble (% w/w) | | | | | | | |
| Ratio of | 44.00 | 168.00 | 7.00 | 21.00 | 8.00 | 22.00 | 1.00 |
| inventive comp. | | | | | | | |
| as compared to | | | | | | | |
| Xifaxan ® | | | | | | | |

TABLE 6B
Percent Soluble Drug in SIF

| | Phosphate Buffer | | SIF | |
|---|---|---|---|---|
| | 5256- | Xifaxan ® | 5256- | |
| | 34E | powder | 34E | Xifaxan ® |
| Ingredients | % w/w | | | |
| Rifaximin | 10.0 | 200 mg | 10.0 | Colloidal |
| Polyoxyl 40 | 35.0 | — | 35.0 | silicon dioxide, |
| Hydrogenated | | | | disodium |
| castor oil | | | | edetate, |
| Diethyl | 27.5 | — | 27.5 | glycerol |
| sebacate | | | | palmitostearate, |
| Diethylene | 27.5 | — | 27.5 | hypromellose |
| glycol | | | | |
| monoethyl | | | | |
| ether | | | | |
| Butylated | — | — | — | |
| hydroxytoluene | | | | |
| Total | 100.0 | 218.00 | 100.0 | |
| Percent of drug | 0.0021 | 0.0003 | 0.0057 | 0.001 |
| soluble (% w/w) | | | | |
| Ratio of | 7.00 | 1.00 | 6.00 | 1.00 |
| inventive | | | | |
| comp. as | | | | |
| compared to | | | | |
| Xifaxan ® | | | | |

The effects from the inclusion of certain additives such as butylated hydroxytoluene (BHT), crystallization inhibitor (CI), and bile acid are shown in Table 7. Similar to the above, the inventive compositions in Table 7 comprised 1 gram total, each comprising 100 mg of rifaximin.

TABLE 7
Effect of Additives

| | No | | | | No Bile | Bile | |
|---|---|---|---|---|---|---|---|
| | BHT | BHT | No CI | CI | acid | acid | |
| | 5256- | 5256- | 5256- | 5256- | 5246- | 5256- | Xifaxan |
| | 34E | 34E | 35A | 35A | 34E | 34E | powder |
| Ingredients | % w/w | | | | | | |
| Rifaximin | 10.0 | 10.0 | 10.0 | 10.0 | 10.0 | 10.0 | 200 mg |
| Polyoxyl 40 | 35.0 | 34.96 | 34.96 | 29.962 | 35.0 | 34.8 | — |
| hydrogenated castor | | | | | | | |
| oil | | | | | | | |
| Diethyl Sebacate | 27.5 | 27.5 | 27.5 | 27.5 | 27.5 | 27.5 | — |
| Diethylene glycol | 27.5 | 27.5 | 27.5 | 27.5 | 27.5 | 27.5 | — |
| monoethyl ether | | | | | | | |
| Butylated | — | 0.038 | 0.038 | 0.038 | — | — | — |
| hydroxytoluene | | | | | | | |
| Isooctyl acrylate/ | — | — | — | 5.0 | — | — | — |
| acrylamide/vinyl | | | | | | | |
| acetate copolymer | | | | | | | |
| (Kollidone ® VA | | | | | | | |
| 64) | | | | | | | |
| Glyceryl distearate | — | — | — | — | — | — | 18 mg |
| Cholic acid | — | — | — | — | — | 0.2 | — |
| Total | 100.0 | 100.0 | 100.0 | 100.0 | 100.0 | 100.0 | 218.00 |
| Percent of drug | 0.0021 | 0.0024 | 0.0024 | 0.0016 | 0.0021 | 0.0014 | 0.0003 |
| soluble (% w/w) | | | | | | | |
| Ratio of inventive | 7.00 | 8.00 | 8.00 | 5.00 | 7.00 | 5.00 | 1.00 |
| comp. as compared | | | | | | | |
| to Xifaxan | | | | | | | |

B. Prototype Compatibility with Gelatin Shells

The compatibility of certain inventive compositions with gelatin shell matrix was investigated. Capsules were size 1, VWR catalogue number 70102. The vials were then subjected to various temperature conditions for up to 5 weeks. Capsules were then evaluated for physical changes (softening or hardening of capsule shell). Results from this study are shown in Table 8 below. The inventive compositions in Table 7 comprised 1 gram total, each comprising 50 mg or 100 mg of rifaximin.

TABLE 8

| | 5256- | 5394- | 5256- | 5394- | 5256- | 5394- |
|---|---|---|---|---|---|---|
| | 27C | 58A | 34E | 57A | 35A | 60A |
| Ingredients | % w/w | | | | | |
| Rifaximin | 10.0 | 5.00 | 10.0 | 5.00 | 10.0 | 5.00 |
| Castor oil | 10.0 | 15.00 | — | — | — | — |
| Glyceryl caprylate | 12.0 | 12.00 | — | — | — | — |
| Polysorbate 80 | 33.0 | 33.00 | — | — | — | — |
| Polyoxyl 40 | 34.96 | 34.96 | 35.0 | 19.96 | 34.96 | 39.96 |
| hydrogenated castor | | | | | | |
| Diethyl sebacate | — | — | 27.5 | 37.50 | 27.5 | 27.50 |
| Diethylene glycol | — | — | 27.5 | 37.50 | 27.5 | 27.50 |
| monoethyl ether | | | | | | |
| butylated | 0.038 | 0.038 | — | 0.038 | 0.038 | 0.038 |
| hydroxytoluene | | | | | | |
| Total | 100.00 | 100.00 | 100.00 | 100.00 | 100.00 | 100.00 |
| Percent of drug | 0.0131 | 0.0503 | 0.0021 | 0.0064 | 0.0024 | 0.0065 |
| soluble (% w/w) | | | | | | |
| Ratio of inventive | 44.00 | 168.00 | 7.00 | 21.00 | 8.00 | 22.00 |
| comp. as compared | | | | | | |
| to Xifaxan ® | | | | | | |
| Hard gel | Yes | Yes | Yes | Yes | Yes | Yes |
| compatibility | | | | | | |
| Soft gel | TBD | Yes | No | No | No | No |
| compatibility | | | | | | |

As shown above, inventive compositions significantly improve the solubility of rifaximin under conditions similar to those present in vivo. In some cases, this effect represents over a 150-fold increase in percent soluble rifaximin when compared to commercially available Xifaxan®.

### 3. Additional Formulations

Additional formulations are shown below in Table 9.

TABLE 9

| | 5507- | 5425- | 5425- | 5425- | 5425- | |
|---|---|---|---|---|---|---|
| | 65A | 68A | 70A | 70B | 70C | A |
| Ingredients | % wt/wt | | | | | |
| Rifaximin | 10.0 | 2.5 | 10.0 | 5.0 | 2.5 | 2.5 |
| Castor oil | 10.0 | 17.5 | — | — | — | 10.0 |
| Polyoxyl 40 hydrogenated castor | 34.90 | 34.962 | 34.96 | 39.96 | 42.46 | 35.0 |
| oil (RH-40) | | | | | | |
| Glyceryl caprylate type I | 12.0 | 12.0 | — | — | — | 12.0 |
| Polysorbate (Tween) 80 | 33.0 | 33.0 | — | — | — | 40.29 |
| Butylated Hydroxytoluene | 0.05 | 0.038 | 0.038 | 0.038 | 0.038 | 0.1 |
| (BHT) | | | | | | |
| Citric acid | 0.05 | — | — | — | — | 0.01 |
| Ascorbyl Palmitate | — | — | — | — | — | 0.1 |
| Diethyl Sebacate | — | — | 16.5 | 16.5 | 16.5 | — |
| Diethylene glycol monoethyl | — | — | 38.5 | 38.5 | 38.5 | — |
| ether | | | | | | |
| Total | 100.0 | 100.0 | 100.0 | 100.0 | 100.0 | 100.0 |

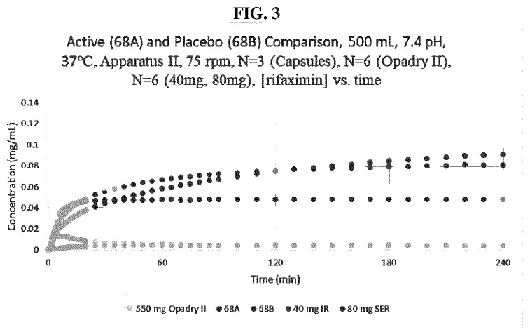

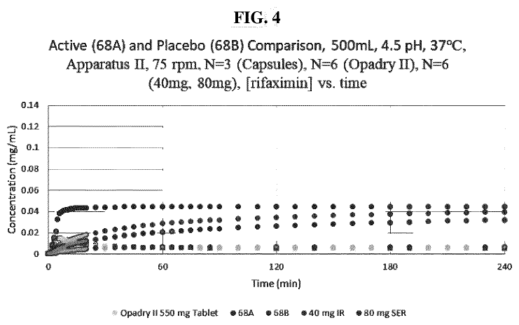

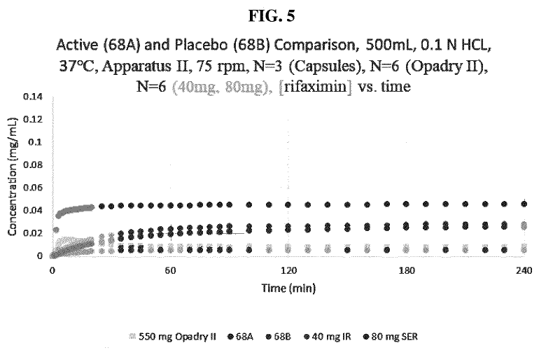

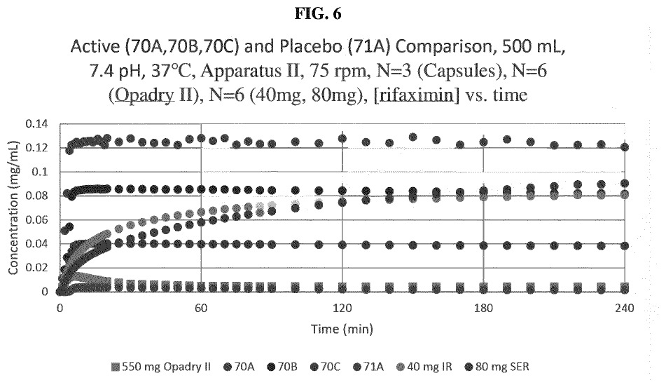

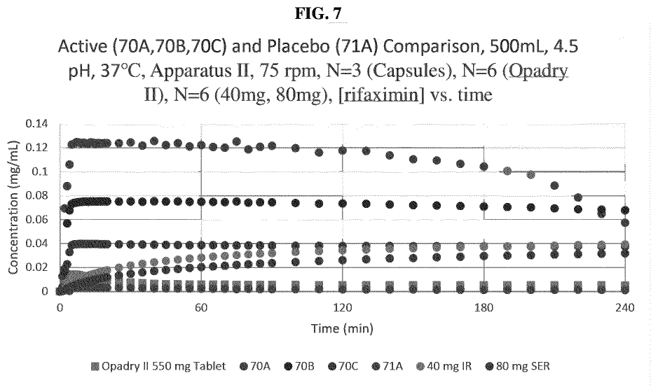

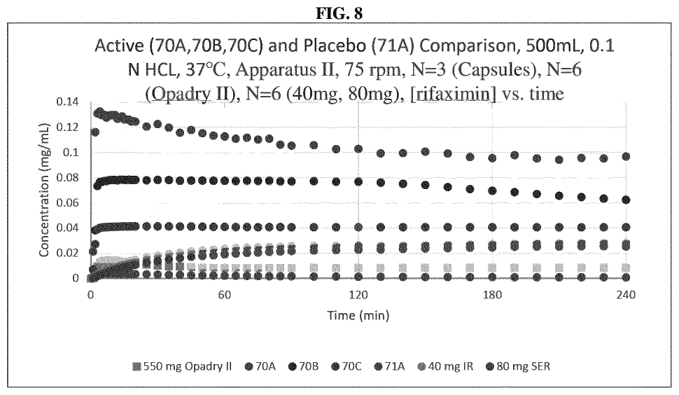

Compositions may be placed in non-enteric coated capsules such as Quali-G, Size 4, 140 mg fill. Dissolution results for 5425-68A, 5425-70A, 5425-70B, and 5425-70C under various pH conditions are shown in FIGS. 3-8, as compared to previously disclosed 40 mg rifaximin IR and 80 mg rifaximin SER solid dispersion compositions (see WO 2018/064472) and Xifaxan 550 mg.

### 4. Anti-Bacterial Activity

The antibacterial properties of representative compositions were examined as set forth herein.

Organisms were prepared by inoculating the surface of Soybean-Casein Digest Agar (TSA) plates, incubated at 30 to 35° C. for 18 to 24 hours. Following the incubation period, the plates were washed with sterile Serological Saline Solution to harvest the microorganisms used and dilutions with Saline were made, plated on TSA and incubated at 30 to 35° C. for 18-24 hours to determine the concentration. The inoculum level was then adjusted to 10\hat{ }cfW mL for use as a stock suspension. Stock suspensions were well mixed and homogenized at each inoculation interval.

The following microorganisms were used in this Kill Time Study Escherichia coli ATCC 8739, Shigella flexneri ATCC 12022, and Salmonella choleraesuis ATCC 10708. Positive controls were performed at initiation and completion by pour plating to enumerate inoculum levels and verify culture purity during testing and Negative controls were performed to establish sterility of media, reagents, and materials used at initiation. Neutralizer Suitability using Modified Letheen Broth (MLB) was performed concurrently with Kill Time testing to confirm the recovery of <100 CFU of the test organism in the subculture media in the presence of product.

1.0 gram of formula (5507-65A) was weighted. In a separate container, a mixture of 70 ml 0.1 N Hydrochloric acid (pH 1.1) and 30 ml of 0.20 M tribasic sodium phosphate was made and the pH was adjusted to about 6.8. The mixture was added to the formula and agitated for 1 hour at room temperature. Then the mixture was filtered through micron filter to remove any precipitate and to collect the filtrate. Duplicate 10 mL containers for each treated specimen or material concentration was prepared, equilibrated to ±2° C., and 0.1 mL of inoculum was added to each container to achieve a final concentration of 10\hat{ }CFU/mL into the product.

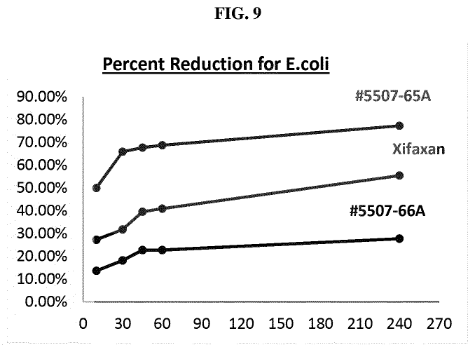

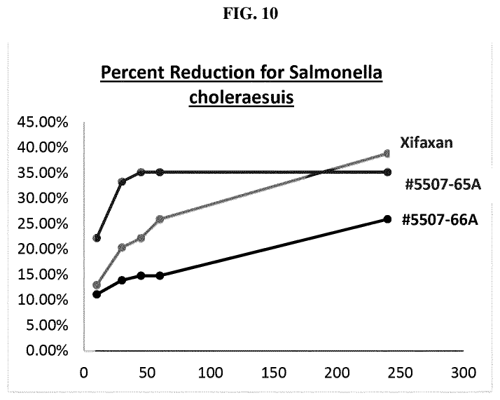

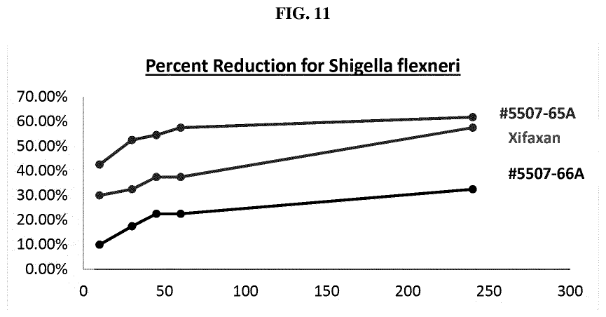

Serial dilutions from each replicate were made at intervals of 10 minutes, 30 minutes, 45 minutes, 1 hour, 2 hours, 3 hours, and 4 hours using lml of the inoculated test product into 9 ml MLB from 1:10 to 1:1000000. Subsequently, 1 mL from each dilution was pour plated with TSA in duplicate, incubated at 30 to 35° C. for 48 hours. After the incubation period, all plates were counted to determine the number of microorganisms, results are averaged and reported as log reductions. A placebo control (5507-66A) of formulation 5507-65A was prepared in the same manner. The placebo contained no rifaximin and comprised 44.90% wt/wt of polyoxyl 40 hydrogenated castor oil instead of 34.90% wt/wt. A Xifaxan 550 mg tablet was also tested and compared. Results are shown in FIGS. 9-11.

### 5. Stability Studies

A stability study was performed to determine whether rifaximin would degrade in a representative liquid composition described herein during storage. The formulation of Table 10 was stored at 5° C. and at 25° C. (at 60% RH) and inspected at 0.5 and 1.0 month for changes in visual appearance and concentration of rifaximin by LC-MS. The results of such study are shown in Table 11, which indicate that little or no degradation of rifaximin was observed in the representative liquid composition.

TABLE 10

| Ingredients | % w/w |
|---|---|
| Rifaximin | 2.50 |
| Castor oil | 10.00 |
| Polyoxyl 40 Hydrogenated | 35.00 |
| Castor Oil | |
| Glyceryl Caprylate Type I | 12.00 |
| Polysorbate (Tween) 80 | 40.29 |
| Butylated Hydroxytoluene | 0.10 |
| Anhydrous Citric Acid | 0.01 |
| Ascorbyl Palmitate | 0.10 |
| Total | 100.00 |

TABLE 11

| Storage | Time | Description | Rifaximin |
|---|---|---|---|
| Conditions | Point | (Visual) | % LC |
| N/A | Initial | Conforms | 100.20 |
| 5° C. | 0.5 | Conforms | 101.00 |
| | Month | | |
| | 1 M | Conforms | 100.10 |
| 25° C./60% | 0.5 | Conforms | 99.10 |
| RH | Month | | |
| | 1 M | Conforms | 100.00 |

6. Proposed Randomized, Double-Blind, Placebo-Controlled Study to Characterize the Safety, Efficacy, Pharmacokinetics, and Pharmacodynamics of Rifaximin Compositions in Sickle Cell Disease Patients with Vaso-Occlusive Crisis.

The purpose of this study is to evaluate the safety, efficacy, and pharmacokinetics of rifaximin after oral administration of one or more of rifaximin compositions disclosed herein in sickle cell disease (SCD) patients with history of vaso-occlusive crisis (VOC). Any potential pharmacokinetic-pharmacodynamic (PK/PD) relationship between rifaximin systemic exposure and potential biomarkers of microbially-associated induction of VOC will also be evaluated. Previous clinical studies have shown rifaximin treatment (i.e., XIFAXAN® 550 mg) to have potential benefit in reducing the number of VOCs and use of intravenous opioid analgesia (IOA). One possible explanation of this benefit may be due to modulation of intestinal microbial composition in SCD patients. Significant elevation of circulating aged neutrophils (CANs), with high CXCR4 and low CD62L surface expression, has been observed during VOCs and has been implicated in the development of the condition. It is proposed that this may occur in response to increased translocation of intestinal bacteria and bacterial products, which may be controlled with the administration of rifaximin.

This study will evaluate the safety, efficacy, and PK of rifaximin in SCD patients, as well as PK/PD relationships between rifaximin and several putative biomarkers associated with the proposed mechanism.

### Objectives

The primary objective of this study is to assess the efficacy of the disclosed composition(s) in reducing VOCs in SCD patients.

Secondary objectives of this study are: (1) assessment of efficacy of the disclosed composition(s) in reducing subcategories of VOCs in SCD patients; (2) assessment of the disclosed composition(s) impact on IOA usage during VOC; (3) assessment of the disclosed composition(s) impact on outpatient opioid usage; (4) assessment of safety and tolerability of the disclosed composition(s) in SCD patients; and (5) characterization of PK and the PK/PD relationships between the disclosed rifaximin composition tested herein and potential biomarkers of microbially-associated VOCs.

### Endpoints

The primary efficacy endpoint of this study will be measured by the annualized rate of VOCs (overall and leading to healthcare visits).

The secondary efficacy endpoints of this study will be measured by the annualized rate of VOCs by subcategory (overall and leading to healthcare visits); the annualized rate of SCD-associated medical facility visits and/or hospitalization visits; and the duration of SCD-associated medical facility visits and/or hospitalization visits.

The secondary endpoint of impact on IOA use for this study is measured by the annualized rate of days using IOA; the time to readiness-for-discharge from first use of IOA during VOC; cumulative IOA consumption during VOC; and time to discontinuation of IOA use during VOC.

The secondary endpoint of assessment of the rifaximin composition's impact on outpatient opioid usage is measured in MME units.

The secondary endpoint of safety is measured by AEs, vital signs, and clinical labs.

The secondary endpoint of PK (rifaximin and 25-desacetyl rifaximin in plasma) is measured by subjects with intensive PK sampling (Day 1): Cₘₐₓ, Tₘₐₓ, AUCₗₐₛₜ, AUC₀₋₁₂, AUC_{inf}, λz, t_{1/2}, CL/F, Vz/F, MR__{AUCinf}; subjects with intensive PK sampling (Day 29): C_{trough}, C_{max,ss}, T_{max,ss}, AUCₜₐᵤ, C_{ss,av} λz, t_{1/2}, CL/Fₛₛ, Vz/Fₛₛ, R_{AUC}, R_{Cmax}, MR_AUCₜₐᵤ; and subjects with sparse sampling: (Day 1, Day 8 [±1 day], Day 15 [±1 day], Day 29 [±1 day], Month 3, and Month 6): C_{trough}, Cₘₐₓ, AUC.

The secondary PD endpoint is measured by number and change from predose on Day 1 (at Day 8 [±1 day], Day 15 [±1 day], Day 29 [±1 day], Month 3, and Month 6) for total neutrophils and CANs, serum CD62L, urine 3-indoxyl sulfate, LPS, zonulin, serum citrulline, intestinal fatty-acid binding protein (iFABP).

The secondary PK/PD endpoint will be measured by evaluating the PK/PD relationships between rifaximin PK and each PD endpoint.

Proposed exploratory endpoints include use of a FANLTC questionnaire; examination of relative taxonomic abundance of fecal microbiota at baseline (screening window), Day 29, and Month; examination of iFABP levels; evaluation of CAN levels; evaluation of Zonulin levels; and evaluation of serum LPS levels.

### Patient Population

SCD Patients that have experienced at least 1 VOC in the 12 months prior to enrollment.

### Key Inclusion and Exclusion Criteria

Inclusion Criteria:
Give informed consent.
Has SCD of any genotype (HbSS, HbSC, HbS β-thalassemia).
18 to 70 years of age (inclusive) on day of consent.
Experienced at least 1 VOC within the preceding 12 months prior to Screening. Prior VOC should include occurrence of appropriate symptoms, visit to medical facility and/or healthcare professional, receipt of parenteral opioid or NSAID analgesia or oral opioid.
If receiving hydroxyurea or hydroxycarbamide (HU/HC) or erythropoietin stimulating agents, patient must have been receiving treatment for at least 6 months prior to Screening and plan to maintain the same dose and schedule during the study.
Must meet the following lab values at screening:
   Absolute Neutrophil Count ≥1.0×10⁹/L
   Platelets ≥75×10⁹/L
   Hemoglobin (Hgb)≥4.0 g/dL
   Glomerular filtration rate ≥45 mL/min/1.73 m 2 using CKD-EPI formula
   Direct (conjugated) bilirubin ≤2.0×ULN
   Alanine transaminase (ALT)≤3.0×ULN
   INR≥2.0
ECOG performance status ≤2

Exclusion Criteria:
History of stem cell transplant.
Acute VOC ending within 7 days prior to Day 1 dosing.
Received any blood products within 30 days of Day 1 dosing.
Uncontrolled liver disease or renal insufficiency, colitis, or inflammatory bowel disease.
Received active treatment on another investigational trial or has taken penicillin prophylaxis or antibiotics for treatment of infection within 30 days or 5 half-lives of the treatment, whichever is greater, prior to screening.
Significant medical condition that requires hospitalization (other than SCD with VOC) within 2 months prior to screening.
Participating in a chronic transfusion program (pre-planned series of transfusions for prophylactic purposes).
Planning on undergoing an exchange transfusion during the duration of the study; episodic transfusion in response to worsened anemia or VOC is permitted.
Hypersensitivity to rifaximin, rifampin, rifamycin antimicrobial agents, or any components of the rifaximin composition.
Use of therapeutic anticoagulation (prophylactic doses permitted) or antiplatelet therapy (other than aspirin or NSAIDs) within the 10 days prior to Day 1 dosing.
Pregnant or nursing women.
Women of child-bearing potential, defined as all women physiologically capable of becoming pregnant, unless tested negative by serum pregnancy test at screening and agrees to standard prevention methods.
History of drug abuse, documented or in opinion of investigator.
Requirement for use of any medications on the prohibited medications list (CYP3A4 inhibitors/inducers, PPIs, PgP substrates).
Any prior gastrointestinal surgery which has altered the anatomy of the esophagus, stomach, or small/large intestine (exceptions include appendectomy, cholecystectomy, and fundoplication).
Colonoscopy or sigmoidoscopy, or any other use of bowel prep, laxative, or enema, within 30 days prior to Day 1 or plans to undergo such a procedure during the duration of the study.
Any documented history of clinical stroke or intracranial hemorrhage, or an uninvestigated neurologic finding within the 12 months prior to screening. Silent infarct only present on imaging is allowed.
Patients with bleeding disorders.
Planning to undergo a major surgical procedure during the duration of the study
Positive for HIV or other concomitant immunodeficiency.
Active Hepatitis B infection (HBsAg positive). Prior infection but not active (i.e., anti-HBc positive, HBsAg and HBV-DNA negative) is allowed.
Positive for Hepatitis C (HCV RNA). Prior infection with spontaneous resolution or sustained resolution after antiviral treatment (i.e., no detectible HCV RNA) for ≥6 months (with IFN-free treatments) or for ≥12 months (with use of IFN treatment) after cessation of antivirals are allowed.
Malignant disease. Exceptions include malignancies that were treated curatively and have not recurred within 2 years prior to study treatment, completely resected basal cell and squamous cell skin cancers, and any completely resected carcinoma in situ.
Serious mental or physical illness which, in the opinion of the Investigator, would compromise participation in the study.
Any condition which, in the opinion of the Investigator, is likely to interfere with the successful collection of the measurements required for the study.
Resting QTcF≥470 msec at screening.
Cardiac or cardiac repolarization abnormality, including any of the following:
   History of myocardial infarction (MI) angina pectoris, coronary artery bypass graft (CABG), or uncontrolled congestive heart failure within 6 months prior to Day 1.
   Clinically significant cardiac arrhythmias (e.g., ventricular tachycardia), complete left bundle branch block, high-grade AV block (e.g., bifascicular block, Mobitz type II and third-degree AV block).
   Long QT syndrome, family history of idiopathic sudden death or congenital long QT syndrome, or any of the following:
      Risk factors for Torsade de Pointes (TdP) including uncorrected hypokalemia or hypomagnesemia, history of cardiac failure, or history of clinically significant/symptomatic bradycardia.
      Concomitant medications with a known risk of TdP that cannot be discontinued or replaced by safe alternative (within 5 half-lives prior to starting study drug).
      Inability to determine the QTcF interval.
Not able to understand or comply with study instructions and requirements.
For subjects in intensive PK group, subjects with hepatic impairment (Child-Pugh Class A, B, or C) should be excluded.

### Subject Assessments

Efficacy assessments to be made include:
Number of VOCs during treatment and history thereof for 12 months prior to treatment. Crises identified by trial investigators will be adjudicated in a blinded fashion by an independent crisis-review committee.
Number of VOCs by subcategory (uncomplicated pain crisis, acute chest syndrome, hepatic sequestration, splenic sequestration, priapism) during treatment and history thereof for 12 months prior to treatment. Crises identified by trial investigators will be adjudicated in a blinded fashion by an independent crisis-review committee.
Number of SCD-associated hospitalization and ER visits during treatment and history thereof for 12 months prior to treatment.
Date and time for each start and stop of IOA use during VOCs.
Duration of hospitalization during each VOC, date/time of first use of IOA, and date/time of readiness-for-discharge.
Cumulative consumption of IOAs during each VOC during treatment and history thereof for 12 months prior to treatment.
Cumulative time of IOA usage during each VOC.

Safety and tolerability assessments include AEs, vital signs, clinical labs, and ECGs.

Other assessments include:
Functional Analysis of Non-life-Threatening Conditions (FANLTC) questionnaire predose on Day 1, at Day 29, 3 months, and 6 months.
Stool sample for microbiome profiling during screening window, at Day 29 and at 6 Months.

PK assessments will be provided as follows:
For intensive PK Subjects (Day 1 & Day 29): Predose, 0.5, 1, 1.5, 2, 3, 4, 5, 8, and 12 hr post dose (12 hr timepoint should be prior to second dose). For all other scheduled visits, predose and up to 3 additional post-dose samples (TBD by pharmacometrician).
For subjects with Sparse Sampling: Predose and up to 3 additional post-dose samples (TBD by pharmacometrician) on Day 1, Day 8, Day 15, Day 29, Month 3, and Month 6.
For all subjects: During medical facility visit for VOC with estimated time of last dose, when possible.
Dosing diary kept with time of dose recorded.

PD assessments will be provided as follows:
Predose on Day 1, during Day 8, Day 15, Day 29, Month 3, and Month 6 visits, and during medical facility visit for VOC when possible:
   Neutrophil markers: Total Neutrophils (Count and % WBC), CANs (Count and % Neutrophils), Serum CD62L.
   Gut permeability markers: Zonulin, serum citrulline, iFABP.
   Gut bacteria markers: LPS, Urine 3-indoxyl sulfate.

### Data Analysis

The primary efficacy end point is the annual rate of VOC, which will be calculated as follows: total number of adjudicated VOC×365÷(end date−date of randomization+1), with the end date defined as the date of the last dose plus 14 days. The difference in the annual VOC rate for each rifaximin group versus the placebo group will be analyzed with a Wilcoxon rank-sum test, stratified by use of categorized history of crises in the previous year (<5; >=5 POV).

Change from Baseline in rate of VOCs, days using IOA, SCD-associated hospitalization and ER events and duration will be summarized by treatment and compared to placebo.

Time to readiness-for-discharge from first use of IOAs during each VOC and time to discontinuation of IOA use during VOCs will be summarized by treatment group with descriptive statistics and presented as Kaplan-Meier plots.

Cumulative use of IOA consumption during VOCs will be summarized by treatment group with descriptive statistics and compared to placebo.

AEs to be summarized by MedDRA System Organ Class (SOC) and Preferred Term (PT) and reported by treatment group and relationship to treatment. Observed and Change from Baseline in Vital Signs, labs, and ECG parameters (RR, PR, QTcF, QRS)

PK will be evaluated in intensive PK subjects using noncompartmental analysis. A population PK model will be developed using data from all subjects providing quantifiable post-dose samples. Steady-state will be assessed for all subjects using C trough measurements on Day 8, Day 15, and Day 29, and may be simulated using the population PK model.

PD endpoints will be summarized by treatment with quantity and change from baseline at each visit.

Relationship between rifaximin PK parameters and each PD endpoint will be evaluated using ANOVA models. A population PK/PD model may be developed as a separate analysis if warranted.

Change from Baseline in FANLTC questionnaire scores will be summarized by treatment and compared to placebo.

Change from Baseline in intestinal microbiome composition may be summarized by treatment and compared to placebo

The contents of all references (including literature references, issued patents, published patent applications, and co-pending patent applications) cited throughout this application are hereby expressly incorporated herein in their entireties by reference.

### Further Embodiments

Embodiment 1a. A pharmaceutically acceptable composition comprising rifaximin, a hydrogenated castor oil, and at least one additional solubilizing excipient for use in the method of treating sickle cell disease (SCD) in a patient in need thereof.

According to a preferred embodiment a pharmaceutically acceptable composition comprising rifaximin, a hydrogenated castor oil, and at least one additional solubilizing excipient is administering to the patient.

Embodiment 2a. The pharmaceutically acceptable composition for use according to embodiment 1a, wherein the patient is experiencing vaso-occlusive crises (VOCs).

Embodiment 3a. A pharmaceutically acceptable composition comprising rifaximin, a hydrogenated castor oil, and at least one additional solubilizing excipient for use in the method of reducing elevated levels of circulating aged neutrophils (CANs) in a patient in need thereof.

According to a preferred embodiment a pharmaceutically acceptable composition comprising rifaximin, a hydrogenated castor oil, and at least one additional solubilizing excipient is administering to the patient.

Embodiment 4a. A pharmaceutically acceptable composition comprising rifaximin, a hydrogenated castor oil, and at least one additional solubilizing excipient for use in the method of treating vaso-occlusive crises (VOCs) in a patient in need thereof.

According to a preferred embodiment a pharmaceutically acceptable composition comprising rifaximin, a hydrogenated castor oil, and at least one additional solubilizing excipient is administering to the patient.

Embodiment 5a. The pharmaceutically acceptable composition for use according to any one of embodiments 1a to 4a, wherein the hydrogenated castor oil is polyoxyl 60 hydrogenated castor oil or polyoxyl 40 hydrogenated castor oil.

Embodiment 6a. The pharmaceutically acceptable composition for use according to any one of embodiments 1a to 5a, wherein the hydrogenated castor oil is polyoxyl 40 hydrogenated castor oil.

Embodiment 7a. The pharmaceutically acceptable composition for use according to any one of embodiments 1a to 6a, wherein the hydrogenated castor oil is present in an amount ranging from about 25% to about 65% by weight of the composition.

Embodiment 8a. The pharmaceutically acceptable composition for use according to any one of embodiments 1a to 7a, wherein the hydrogenated castor oil is present in an amount ranging from about 25% to about 50% by weight of the composition.

Embodiment 9a. The pharmaceutically acceptable composition for use according to any one of embodiments 1a to 8a, wherein the hydrogenated castor oil is present in an amount ranging from about 30% to about 45% by weight of the composition.

Embodiment 10a. The pharmaceutically acceptable composition for use according to any one of embodiments 1a to 9a, wherein the hydrogenated castor oil is present in an amount ranging from about 35% to about 40% by weight of the composition.

Embodiment 11a. The pharmaceutically acceptable composition for use according to any one of embodiments 1a to 10a, wherein the at least one additional solubilizing excipient is selected from a water-soluble organic solvent, a non-ionic surfactant, a water-insoluble lipid, and long-chain triglycerides, and combinations thereof.

Embodiment 12a. The pharmaceutically acceptable composition for use according to any one of embodiments 1a to 11a, wherein the at least one additional solubilizing excipient is selected from polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, and combinations thereof.

Embodiment 13a. The pharmaceutically acceptable composition for use according to any one of embodiments 1a to 12a, wherein the at least one additional solubilizing excipient is one which allows for a rifaximin saturation solubility of greater than about 10% w/w.

Embodiment 14a. The pharmaceutically acceptable composition for use according to any one of embodiments 1a to 13a, wherein the at least one additional solubilizing excipient is selected from polyethylene glycol 600, glyceryl caprylate, polysorbate 80, castor oil, benzyl alcohol, polyethylene glycol 400, diethylene glycol monoethyl ether, glyceryl monooleate, triethylene glycol, diisopropyl adipate, diethyl sebacate, olelyl alcohol, polysorbate 20, oleic acid, caprylic capric triglycerides, propylene glycol, sesame oil, soybean oil, and corn oil, and combinations thereof.

Embodiment 15a. The pharmaceutically acceptable composition for use according to any one of embodiments 1a to 14a, wherein the at least one additional solubilizing excipient is one which allows for a rifaximin saturation solubility of greater than about 14% w/w.

Embodiment 16a. The pharmaceutically acceptable composition for use according to any one of embodiments 1a to 15a, wherein the at least one additional solubilizing excipient is selected from castor oil, glyceryl caprylate, polysorbate 80, diethyl sebacate, and diethylene glycol monoethyl ether, and combinations thereof.

Embodiment 17a. The pharmaceutically acceptable composition for use according to any one of embodiments 1a to 16a, wherein the at least one additional solubilizing excipient is present in an amount ranging from about 45% to about 65% by weight of the composition.

Embodiment 18a. The pharmaceutically acceptable composition for use according to any one of embodiments 1a to 17a, wherein the at least one additional solubilizing excipient is present in an amount ranging from about 50% to about 65% by weight of the composition.

Embodiment 19a. The pharmaceutically acceptable composition for use according to any one of embodiments 1a to 18a, wherein the at least one additional solubilizing excipient is present in an amount ranging from about 55% to about 65% by weight of the composition.

Embodiment 20a. The pharmaceutically acceptable composition for use according to any one of embodiments 1a to 19a, wherein the at least one additional solubilizing excipient is present in an amount ranging from about 60% to about 65% by weight of the composition.

Embodiment 21a. The pharmaceutically acceptable composition for use according to any one of embodiments 1a to 20a, wherein the at least one additional solubilizing excipient is present in an amount ranging from about 55% to about 60% by weight of the composition.

Embodiment 22a. The pharmaceutically acceptable composition for use according to any one of embodiments 1a to 21a, wherein the at least one additional solubilizing excipient is present in an amount of about 55% by weight of the composition.

Embodiment 23a. The pharmaceutically acceptable composition for use according to any one of embodiments 1a to 22a, wherein the at least one additional solubilizing excipient is present in an amount of about 62% or about 63% by weight of the composition.

Embodiment 24a. The pharmaceutically acceptable composition for use according to any one of embodiments 1a to 23a, wherein the at least one additional solubilizing excipient is a combination of castor oil, glyceryl caprylate, and polysorbate 80.

Embodiment 25a. The pharmaceutically acceptable composition for use according to embodiment 24a, wherein the castor oil is present in an amount ranging from about 5% to about 15% by weight of the composition; the glyceryl caprylate is present in an amount ranging from about 5% to about 15% by weight of the composition; and the polysorbate 80 is present in an amount ranging from about 20% to about 40% by weight of the composition.

Embodiment 26a. The pharmaceutically acceptable composition for use according to embodiments 24a or 25a, wherein the castor oil is present in an amount ranging from about 8% to about 12% by weight of the composition; the glyceryl caprylate is present in an amount ranging from about 10% to about 14% by weight of the composition; and the polysorbate 80 is present in an amount ranging from about 30% to about 35% by weight of the composition.

Embodiment 27a. The pharmaceutically acceptable composition for use according to embodiment 24a, wherein the castor oil is present in an amount ranging from about 10% to about 20% by weight of the composition; the glyceryl caprylate is present in an amount ranging from about 5% to about 15% by weight of the composition; and the polysorbate 80 is present in an amount ranging from about 20% to about 40% by weight of the composition.

Embodiment 28a. The pharmaceutically acceptable composition for use according to embodiments 24a or 27a, wherein the castor oil is present in an amount ranging from about 13% to about 18% by weight of the composition; the glyceryl caprylate is present in an amount ranging from about 10% to about 14% by weight of the composition; and the polysorbate 80 is present in an amount ranging from about 30% to about 35% by weight of the composition.

Embodiment 29a. The pharmaceutically acceptable composition for use according to embodiment 24a, wherein the castor oil is present in an amount ranging from about 5% to about 15% by weight of the composition; the glyceryl caprylate is present in an amount ranging from about 10% to about 15% by weight of the composition; and the polysorbate 80 is present in an amount ranging from about 35% to about 45% by weight of the composition.

Embodiment 30a. The pharmaceutically acceptable composition for use according to embodiments 24a or 29a, wherein the castor oil is present in an amount ranging from about 8% to about 12% by weight of the composition; and the polysorbate 80 is present in an amount ranging from about 38% to about 42% by weight of the composition.

Embodiment 31a. The pharmaceutically acceptable composition for use according to any one of embodiments 1a to 23a, wherein the at least one additional solubilizing excipient is a combination of diethyl sebacate and diethylene glycol monoethyl ether.

Embodiment 32a. The pharmaceutically acceptable composition for use according to embodiment 31a, wherein the diethyl sebacate is present in an amount ranging from about 20% to about 35% by weight of the composition; and the diethylene glycol monoethyl ether is present in an amount ranging from about 20% to about 35% by weight of the composition.

Embodiment 33a. The pharmaceutically acceptable composition for use according to embodiments 31a or 32a, wherein the diethyl sebacate is present in an amount ranging from about 25% to about 30% by weight of the composition; and the diethylene glycol monoethyl ether is present in an amount ranging from about 25% to about 30% by weight of the composition.

Embodiment 34a. The pharmaceutically acceptable composition for use according to embodiment 31a, wherein the diethyl sebacate is present in an amount ranging from about 10% to about 20% by weight of the composition; and the diethylene glycol monoethyl ether is present in an amount ranging from about 30% to about 45% by weight of the composition.

Embodiment 35a. The pharmaceutically acceptable composition for use according to embodiments 31a or 34a, wherein the diethyl sebacate is present in an amount ranging from about 14% to about 18% by weight of the composition; and the diethylene glycol monoethyl ether is present in an amount ranging from about 36% to about 40% by weight of the composition.

Embodiment 36a. The pharmaceutically acceptable composition for use according to any one of embodiments 1a to 35a, wherein the composition further comprises an antioxidant and/or a chelating agent.

Embodiment 37a. The pharmaceutically acceptable composition for use according to any one of embodiments 1a to 36a, wherein the composition further comprises an antioxidant and/or chelating agent selected from the group consisting of ascorbyl palmitate, butylated hydroxyanisole, butylated hydroxytoluene (BHT), potassium metabisulfite, sodium metabisulfite, cysteine, propyl gallate, sodium thiosulfate, vitamin E, 3,4-dihydroxybenzoic acid, and a combination thereof.

Embodiment 38a. The pharmaceutically acceptable composition for use according to any one of embodiments 1a to 37a, wherein the composition further comprises one or more of BHT and citric acid.

Embodiment 39a. The pharmaceutically acceptable composition for use according to any one of embodiments 1a to 38a, wherein the composition further comprises one or more of BHT and citric acid, each in an amount ranging from about 0.01% to about by weight of the composition.

Embodiment 40a. The pharmaceutically acceptable composition for use according to any one of embodiments 1a to 37a, wherein the composition further comprises one or more of BHT, citric acid, and ascorbyl palmitate.

Embodiment 41a. The pharmaceutically acceptable composition for use according to any one of embodiments 1a to 37a and 40a, wherein the composition further comprises one or more of BHT, citric acid, and ascorbyl palmitate, each in an amount ranging from about 0.05% to about 0.15% by weight of the composition.

Embodiment 42a. The pharmaceutically acceptable composition for use according to any one of embodiments 1a to 41a, wherein the rifaximin is present in an amount ranging from about 1.0% to about 15% by weight of the composition.

Embodiment 43a. The pharmaceutically acceptable composition for use according to any one of embodiments 1a to 42a, wherein the rifaximin is present in an amount ranging from about 2.5% to about 15% by weight of the composition.

Embodiment 44a. The pharmaceutically acceptable composition for use according to any one of embodiments 1a to 43a, wherein the rifaximin is present in an amount ranging from about 2.5% to about 12% by weight of the composition.

Embodiment 45a. The pharmaceutically acceptable composition for use according to any one of embodiments 1a to 44a, wherein the rifaximin is present in an amount ranging from about 5% to about 10% by weight of the composition.

Embodiment 46a. The pharmaceutically acceptable composition for use according to any one of embodiments 1a to 45a, wherein the rifaximin is present in an amount of about 5% or about 10% by weight of the composition.

Embodiment 47a. The pharmaceutically acceptable composition for use according to any one of embodiments 1a to 42a, wherein the rifaximin is present in an amount ranging from about 1% to about 5% by weight of the composition.

Embodiment 48a. The pharmaceutically acceptable composition for use according to any one of embodiments 1a to 44a and 47a, wherein the rifaximin is present in an amount of about 2.5% by weight of the composition.

Embodiment 49a. The pharmaceutically acceptable composition for use according to any one of embodiments 1a to 48a, wherein the total amount of rifaximin in the composition is less than about 125 mg.

Embodiment 50a. The pharmaceutically acceptable composition for use according to any one of embodiments 1a to 49a, wherein the total amount of rifaximin in the composition ranges from about 1 mg to about 125 mg.

Embodiment 51a. The pharmaceutically acceptable composition for use according to any one of embodiments 1a to 50a, wherein the total amount of rifaximin in the composition ranges from about 1 mg to about 50 mg.

Embodiment 52a. The pharmaceutically acceptable composition for use according to any one of embodiments 1a to 51a, wherein the total amount of rifaximin in the composition ranges from about 1 mg to about 25 mg or from about 1 mg to about 10 mg.

Embodiment 53a. The pharmaceutically acceptable composition for use according to any one of embodiments 1a to 52a, wherein the total amount of rifaximin in the composition is about 1 mg or about 5 mg.

Embodiment 54a. The pharmaceutically acceptable composition for use according to embodiment 1a, wherein the composition comprises about 10% rifaximin, about 10% castor oil, about 12% glyceryl caprylate, about 33% polysorbate 80, and about 35% polyoxyl 40 hydrogenated castor oil, by weight of the composition.

Embodiment 55a. The pharmaceutically acceptable composition for use according to embodiment 1a, wherein the composition comprises about 10% rifaximin, about 35% polyoxyl 40 hydrogenated castor oil, about 27.5% diethyl sebacate, and about 27.5% diethylene glycol monoethyl ether, by weight of the composition.

Embodiment 56a. The pharmaceutically acceptable composition for use according to embodiment 1a, wherein the composition comprises about 5% rifaximin, about 15% castor oil, about 12% glyceryl caprylate, about 33% polysorbate 80, and about 35% polyoxyl 40 hydrogenated castor oil, by weight of the composition.

Embodiment 57a. The pharmaceutically acceptable composition for use according to embodiment 1a, wherein the composition comprises about 10% rifaximin, about 40% polyoxyl 40 hydrogenated castor oil, about 16.5% diethyl sebacate, and about 38.5% diethylene glycol monoethyl ether, by weight of the composition.

Embodiment 58a. The pharmaceutically acceptable composition for use according to embodiment 1a, wherein the composition comprises about 2.5% rifaximin, about 10% castor oil, about 12% glyceryl caprylate, about 40% polysorbate 80, and about 35% polyoxyl 40 hydrogenated castor oil, by weight of the composition.

Embodiment 59a. The pharmaceutically acceptable composition for use according to any one of embodiments 54a to 58a, wherein the composition further comprises less than from about 0.03% BHT to about 0.06% BHT by weight of the composition.

Embodiment 60a. The pharmaceutically acceptable composition for use according to any one of embodiments 55a to 59a, wherein the composition further comprises less than from about 0.03% BHT to about 0.04% BHT by weight of the composition.

Embodiment 61a. The pharmaceutically acceptable composition for use according to any one of embodiments 54a to 58a, wherein the composition further comprises from about 0.05% BHT to about 0.15% BHT by weight of the composition.

Embodiment 62a. The pharmaceutically acceptable composition for use according to any one of embodiments 54a to 58a and 61a, wherein the composition further comprises from about 0.1% BHT by weight of the composition.

Embodiment 63a. The pharmaceutically acceptable composition for use according to any one of embodiments 54a to 62a, wherein the composition further comprises from about 0.05% to about 0.15% ascorbyl palmitate by weight of the composition.

Embodiment 64a. The pharmaceutically acceptable composition for use according to any one of embodiments 54a to 63a, wherein the composition further comprises from about 0.1% ascorbyl palmitate by weight of the composition.

Embodiment 65a. The pharmaceutically acceptable composition for use according to any one of embodiments 1a to 64a, wherein the composition is a liquid composition.

Embodiment 66a. The pharmaceutically acceptable composition for use according to any one of embodiments 1a to 65a, wherein the composition is present in a soft or hard capsule.

Embodiment 67a. The pharmaceutically acceptable composition for use according to any one of embodiments 1a to 66a, wherein the composition is present in a gelatin capsule.

Embodiment 68a. The pharmaceutically acceptable composition for use according to embodiments 1a to 67a, wherein further to the pharmaceutically acceptable composition an additional SCD therapeutic agent is administered to the patient.

Embodiment 69a. The pharmaceutically acceptable composition for use according to embodiment 68a, wherein the additional SCD therapeutic agent comprises hydroxyurea, L-glutamine, hydroxycarbamide, an erythropoietin stimulating agent, an opioid analgesic, or a combination thereof.

Embodiment 70a. The pharmaceutically acceptable composition for use according to embodiment 69a, wherein the opioid analgesic comprises morphine, codeine, hydrocodone, hydromorphone, methadone, tramadol, oxycodone, tapentadol, fentanyl, or a combination thereof.

Embodiment 71a. The pharmaceutically acceptable composition for use according to any one of embodiments 4a to 70a, wherein the use in the method of treating vaso-occlusive crises (VOCs) in the patient in need thereof comprises alleviating one or more symptoms of VOCs in the patient.

Embodiment 72a. The pharmaceutically acceptable composition for use according to any one of embodiments 4a to 70a, wherein the use in the method of treating vaso-occlusive crises (VOCs) in the patient in need thereof comprises reducing or preventing the occurrence of VOCs in the patient.

Embodiment 73a. The pharmaceutically acceptable composition for use according to any one of embodiments 4a to 70a, wherein the use in the method of treating vaso-occlusive crises (VOCs) in the patient in need thereof comprises reducing the duration or severity of VOCs in the patient.

Embodiment 74a. The pharmaceutically acceptable composition for use according to any one of embodiments 4a to 70a, wherein the use in the method of treating vaso-occlusive crises (VOCs) in the patient in need thereof comprises mediating or otherwise reducing the patient's opioid usage during VOCs.

Embodiment 75a. The pharmaceutically acceptable composition for use according to any one of embodiments 1a to 74a, wherein the pharmaceutical composition is administered to the subject QD, BID, TID, or QID.

Embodiment 76a. The pharmaceutically acceptable composition for use according to any one of embodiments 1a to 75a, wherein the pharmaceutical composition is administered to the subject BID.

Embodiment 77a. The pharmaceutically composition comprising rifaximin, a hydrogenated castor oil, and at least one additional solubilizing excipient for use in the treatment of sickle cell disease (SCD).

Embodiment 78a. The pharmaceutically composition for use according to embodiment 77a, wherein the patient is experiencing vaso-occlusive crises (VOCs).

Embodiment 79a. The pharmaceutically composition comprising rifaximin, a hydrogenated castor oil, and at least one additional solubilizing excipient for use in the treatment of reducing elevated levels of circulating aged neutrophils (CANs).

Embodiment 80a. The pharmaceutically composition comprising rifaximin, a hydrogenated castor oil, and at least one additional solubilizing excipient for use in the treatment of vaso-occlusive crises (VOCs).

Embodiment 81a. The pharmaceutically composition for use according to any one of embodiments 77a to 80a, wherein the composition is a liquid composition.

Embodiment 82a. The pharmaceutically composition for use according to any one of embodiments 77a to 81a, wherein the composition is present in a soft or hard capsule.

Embodiment 83a. The pharmaceutically composition for use according to any one of embodiments 77a to 82a, wherein the composition is present in a gelatin capsule.

Embodiment 84a. The pharmaceutically composition for use according to any one of embodiments 77a to 83a, further comprising an additional SCD therapeutic agent.

Embodiment 85a. The pharmaceutically composition for use according to embodiment 84a, wherein the additional SCD therapeutic agent comprises hydroxyurea, L-glutamine, hydroxycarbamide, an erythropoietin stimulating agent, an opioid analgesic, or a combination thereof.

Embodiment 86a. The pharmaceutically composition for use according to embodiment 85a, wherein the opioid analgesic comprises morphine, codeine, hydrocodone, hydromorphone, methadone, tramadol, oxycodone, tapentadol, fentanyl, or a combination thereof.

1. A method of treating sickle cell disease (SCD) in a patient in need thereof comprising administering to the patient a pharmaceutically acceptable composition comprising rifaximin, a hydrogenated castor oil, and at least one additional solubilizing excipient.

2. (canceled)

3. A method of reducing elevated levels of circulating aged neutrophils (CANs) in a patient in need thereof comprising administering to the patient a pharmaceutically acceptable composition comprising rifaximin, a hydrogenated castor oil, and at least one additional solubilizing excipient.

4. A method of treating vaso-occlusive crises (VOCs) in a patient in need thereof comprising administering to the patient a pharmaceutically acceptable composition comprising rifaximin, a hydrogenated castor oil, and at least one additional solubilizing excipient.

5. The method of claim 1, wherein the hydrogenated castor oil is polyoxyl 60 hydrogenated castor oil or polyoxyl 40 hydrogenated castor oil.

6. (canceled)

7. The method of claim 1, wherein the hydrogenated castor oil is present in an amount ranging from about 25% to about 65% by weight of the composition.

8-10. (canceled)

11. The method of claim 1, wherein the at least one additional solubilizing excipient is selected from a water-soluble organic solvent, a non-ionic surfactant, a water-insoluble lipid, and long-chain triglycerides, and combinations thereof.

12-14. (canceled)

15. The method of claim 1, wherein the at least one additional solubilizing excipient is one which allows for a rifaximin saturation solubility of greater than about 14% w/w.

16. (canceled)

17. The method of claim 1, wherein the at least one additional solubilizing excipient is present in an amount ranging from about 40% to about 65% by weight of the composition.

18-24. (canceled)

25. The method of claim 1, wherein the at least one additional solubilizing excipient is a combination of castor oil, glyceryl caprylate, and polysorbate 80.

26. The method of claim 25, wherein
the castor oil is present in an amount ranging from about 5% to about 15% by weight of the composition;
the glyceryl caprylate is present in an amount ranging from about 5% to about 15% by weight of the composition; and
the polysorbate 80 is present in an amount ranging from about 20% to about 40% by weight of the composition.

27. (canceled)

28. The method of claim 25, wherein
the castor oil is present in an amount ranging from about 10% to about 20% by weight of the composition;
the glyceryl caprylate is present in an amount ranging from about 5% to about 15% by weight of the composition; and
the polysorbate 80 is present in an amount ranging from about 20% to about 40% by weight of the composition.

29. (canceled)

30. The method of claim 25, wherein
the castor oil is present in an amount ranging from about 5% to about 15% by weight of the composition;
the glyceryl caprylate is present in an amount ranging from about 10% to about 15% by weight of the composition; and
the polysorbate 80 is present in an amount ranging from about 35% to about 45% by weight of the composition.

31. (canceled)

32. The method of claim 1, wherein the at least one additional solubilizing excipient is a combination of diethyl sebacate and diethylene glycol monoethyl ether.

33. The method of claim 32, wherein
the diethyl sebacate is present in an amount ranging from about 20% to about 35% by weight of the composition; and
the diethylene glycol monoethyl ether is present in an amount ranging from about 20% to about 35% by weight of the composition.

34. (canceled)

35. The method of claim 32, wherein
the diethyl sebacate is present in an amount ranging from about 10% to about 20% by weight of the composition; and
the diethylene glycol monoethyl ether is present in an amount ranging from about 30% to about 45% by weight of the composition.

36-38. (canceled)

39. The method of claim 1, wherein the composition further comprises one or more of BHT and citric acid.

40-42. (canceled)

43. The method of claim 1, wherein the rifaximin is present in an amount ranging from about 1.0% to about 15% by weight of the composition.

44-49. (canceled)

50. The method of claim 1, wherein the total amount of rifaximin in the composition is less than about 125 mg.

51-54. (canceled)

55. The method of claim 1, wherein the composition comprises
about 10% rifaximin, about 10% castor oil, about 12% glyceryl caprylate, about 33% polysorbate 80, and about 35% polyoxyl 40 hydrogenated castor oil, by weight of the composition; or
about 10% rifaximin, about 35% polyoxyl 40 hydrogenated castor oil, about 27.5% diethyl sebacate, and about 27.5% diethylene glycol monoethyl ether, by weight of the composition; or
about 5% rifaximin, about 15% castor oil, about 12% glyceryl caprylate, about 33% polysorbate 80, and about 35% polyoxyl 40 hydrogenated castor oil, by weight of the composition; or
about 10% rifaximin, about 40% polyoxyl 40 hydrogenated castor oil, about 16.5% diethyl sebacate, and about 38.5% diethylene glycol monoethyl ether, by weight of the composition; or
about 2.5% rifaximin, about 10% castor oil, about 12% glyceryl caprylate, about 40% polysorbate 80, and about 35% polyoxyl 40 hydrogenated castor oil, by weight of the composition.

60-66. (canceled)

67. The method of claim 1, wherein the composition is a liquid composition.

68-88. (canceled)
